(19) United States
(12) Patent Application Publication
(10) Pub. No.: US 2023/0250131 A1
(43) Pub. Date: Aug. 10, 2023

(54) HYDROXYSTEROID COMPOUNDS

(71) Applicant: Epirium Bio Inc., San Diego, CA (US)

(72) Inventors: Ajay Ramchandra Tilekar, Maharashtra (IN); Bhavesh Dave, Maharashtra (IN)

(21) Appl. No.: 18/165,776

(22) Filed: Feb. 7, 2023

Related U.S. Application Data
(60) Provisional application No. 63/307,915, filed on Feb. 8, 2022.

(51) Int. Cl. C07J 73/00; C07J 5/00

(52) U.S. Cl. CPC C07J 73/005; C07J 5/0015; C07J 5/0053; C07B 2200/05

(57) ABSTRACT

The present application discloses derivatives of hydroxysteroids, pharmaceutically acceptable salts, stereoisomers or tautomers thereof. The compounds and compositions described herein can be used in therapy.

### CROSS-REFERENCE TO RELATED APPLICATIONS

This application claims the benefit of provisional U.S. Appl. Ser. No. 63/307,915, filed Feb. 8, 2022, which is incorporated herein in its entirety for all purposes.

### FIELD OF THE INVENTION

The present disclosure relates generally to derivatives of hydroxysteroid compounds. Process for preparation of the compounds, compositions comprising the compounds and methods of use are also provided.

### BACKGROUND OF THE INVENTION

Hydroxysteroids are hydroxylated compounds with a sterol structure and are known to be produced in cells when the mitochondria are exposed to high levels of endogenous H₂O₂ which then acts via the mitochondrial enzyme, 11β-hydroxylase, to hydroxylate a variety of steroids, including cholesterol, pregnenolone, progesterone, and others. Hydroxylation can occur in numerous positions, including the 7, 16, and 11 positions. However, hydroxysteroids that have functions on mitochondrial activity are currently limited and there remains a need for novel compounds for the treatment of diseases and/or conditions associated with mitochondrial dysfunction and toxicity.

### SUMMARY OF THE INVENTION

Provided herein are compounds, salts thereof, pharmaceutical compositions of the foregoing and methods of making and using the same.

In one aspect, provided is a compound of formula (II):

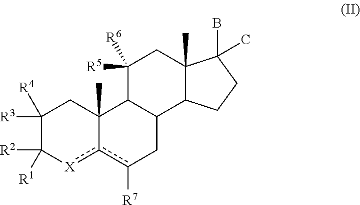

or a pharmaceutically acceptable salt, stereoisomer or tautomer thereof, wherein R¹, R², R³, R⁴, R⁵, R⁶, R⁷, X, B and C are as detailed herein.

Also provided herein is a pharmaceutical composition comprising a compound of any formula herein, including formula (II), a pharmaceutically acceptable salt, stereoisomer or tautomer thereof, and a pharmaceutically acceptable carrier.

In yet another aspect, the compounds described herein have 5′ AMP-activated protein kinase (AMPK) activities and can be useful in AMPK activation.

### DETAILED DESCRIPTION

The present application discloses hydroxysteroid compounds, pharmaceutically acceptable salts, stereoisomers or tautomers thereof, and processes for preparation thereof. The compounds and compositions described herein can be used in therapy.

### Definitions

As used herein and in the appended claims, the singular forms “a”, “an” and “the” include plural forms, unless the context clearly dictates otherwise.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to a subject (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

“Pharmaceutically acceptable salts” are those salts which retain at least some of the biological activity of the free (non-salt) compound and which can be administered as drugs or pharmaceuticals to a subject. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

Asymmetric centers exist in the compounds disclosed herein. These centers are designated by the symbols “R” or “S,” depending on the configuration of substituents around the chiral carbon atom. It should be understood that stereochemical isomeric forms, including diastereomeric, enantiomeric, and epimeric forms as well as d-isomers and l-isomers, and mixtures thereof are encompassed. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. All cis, trans, syn, anti, entgegen (E), and zusammen (Z) isomers as well as the appropriate mixtures thereof are included. Additionally, compounds may exist as tautomers; all tautomeric isomers are provided. Additionally, the compounds disclosed herein can exist in unsolvated as well as solvated forms with pharmaceutically acceptable solvents such as water, ethanol, and the like. In general, the solvated forms are considered equivalent to the unsolvated forms.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

### Compounds

In another aspect, provided is a compound of formula (II):

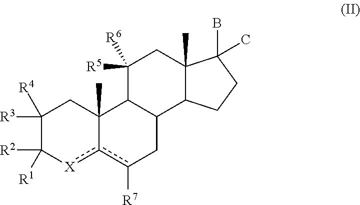

or a pharmaceutically acceptable salt, stereoisomer or tautomer thereof, wherein:

[symbol] is independently either a single bond or a double bond, provided that adjacent double bonds are not allowed;

R¹ is ═O, OH, or OSO₃H;

R² is absent, H or deuterium;

R³ and R⁴ are independently H or deuterium;

R⁵ is OH, H, or OSO₃H;

R⁶ is H, deuterium or CH₃;

R⁷ is H or deuterium;

X is CH, CD, CH₂ or NR⁸, wherein R⁸ is C₁-C₆ alkyl;

B is selected from the group consisting of —COR⁹, C₁-C₆ alkyl, and 5-6 membered heterocycloalkyl, wherein R⁹ is CH₃ or CD₃, the C₁-C₆ alkyl is further substituted with OH, D or both, and the 5-6 membered heterocycloalkyl is optionally substituted with CH₃; and

C is H or deuterium;

provided that when X is CH, CD, or CH₂, then B is —COR⁹, wherein R⁹ is CH₃ or CD₃, and the compound is not selected from the group consisting of:
1-((3S,8S,9S,10R,11S,13S,14S,17S)-3,11-dihydroxy-10,13-dimethyl-2,3,4,7,8,9,10,11,12,13,14,15,16,17-tetradecahydro-1H-cyclopenta[a]phenanthren-17-yl)ethanone,
1-((3S,8S,9S,10R,11S,13S,14S,17S)-3,11-dihydroxy-10,13-dimethyl-2,3,6,7,8,9,10,11,12,13,14,15,16,17-tetradecahydro-1H-cyclopenta[a]phenanthren-17-yl)ethanone,
(8S,9S,10R,11S,13S,14S,17S)-17-acetyl-11-hydroxy-10,13-dimethyl-6,7,8,9,10,11,12,13,14,15,16,17-dodecahydro-1H-cyclopenta[a]phenanthren-3(2H)-one,
1-((3S,8S,9S,10S,11S,13S,14S,17S)-3,11-dihydroxy-10,13-dimethylhexadecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl)ethan1-one,
(10R,11S,13S,17S)-17-acetyl-11-hydroxy-10,13-dimethyl-1,2,6,7,8,9,10,11,12,13,14,15,16,17-tetradecahydro-3H-cyclopenta[a]phenanthren-3-one,
(10S,11S,13S,17S)-17-acetyl-11-hydroxy-10,13-dimethylhexadecahydro-3H-cyclopenta[a]phenanthren-3-one,
(10R,11S,13S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10S,11S,13S,17S)-3,11-dihydroxy-10,13-dimethylhexadecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3-d)ethan-1-one,
1-((3S,10R,11S,13S,17S)-3,11-dihydroxy-10,11,13-trimethyl-2,3,4,7,8,9,10,11,12,13,14,15,16,17-tetradecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-11-d)ethan-1-one,
(8S,9S,10R,11S,13S,14S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3,11-d₂)ethan-1-one,
1-((8S,9S,10R,11S,13 S,14S)-3,11-dihydroxy-10,13-dimethyl-2,3,4,7,8,9,10,11,12,13,14,15,16,17-tetradecahydro-1H-cyclopenta[a]phenanthren-17-yl)ethanone,
(8S,9S,10R,11S,13S,14S)-17-acetyl-1-hydroxy-10,13-dimethyl-6,7,8,9,10,11,12,13,14,15,16,17-dodecahydro-1H-cyclopenta[a]phenanthren-3(2H)-one,
(3S,10S,11S,13S,17S)-17-((R)-1-hydroxyethyl)-10,13-dimethylhexadecahydro-1H-cyclopenta[a]phenanthrene-3,11-diol,
(3S,10R,11S,13S,17S)-17-((R)-1-hydroxyethyl)-10,13-dimethyl-2,3,6,7,8,9,10,11,12,13,14,15,16,17-tetradecahydro-1H-cyclopenta[a]phenanthrene-3,11-diol,
(10R,11S,13S,17S)-11-hydroxy-17-((R)-1-hydroxyethyl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxypropan-2-yl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one,
(3S,10R,11S,13S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthrene-3,11-diol,
(3S,10R,11S,13S,17S)-17-((R)-1-hydroxyethyl)-10,13-dimethyl-2,3,4,7,8,9,10,11,12,13,14,15,16,17-tetradecahydro-1H-cyclopenta[a]phenanthrene-3,11-diol,
(3S,10R,11S,13R,17S)-17-ethyl-10,13-dimethyl-2,3,4,7,8,9,10,11,12,13,14,15,16,17-tetradecahydro-1H-cyclopenta[a]phenanthrene-3,11-diol, and
Trimethylammonium (3S,8S, 9S, 10R,11S,13S,17S)-17-acetyl-11-hydroxy-10,13-dimethyl-2,3,4,7,8,9,10,11,12,13,14,15,16,17-tetradecahydro-1H-cyclopenta[a]phenanthren-3-yl sulfate,
   or a pharmaceutically acceptable salt, stereoisomer or tautomer thereof.

In some embodiments, a compound of formula (II) is a compound of formula (II-a),

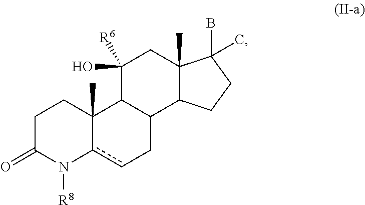

or a pharmaceutically acceptable salt, stereoisomer or tautomer thereof.

In some embodiments, X is CH, CD, CH₂ or NR⁸, wherein R⁸ is C₁-C₆ alkyl. In some embodiments, X is NR⁸, wherein R⁸ is C₁-C₆ alkyl. In some embodiments, R⁸ is ethyl. In some embodiments, R⁸ is propyl. In some embodiments, X is CH, CD, or CH₂.

In some embodiments, R¹ is ═O, OH, or OSO₃H. In some embodiments, R² is absent, H or deuterium. In some embodiments, R-2- is absent. In some embodiments, R¹ is ═O and R² is absent. In some embodiments, R¹ is OH. In some embodiments, R³ and R⁴ are independently H or deuterium. In some embodiments, R³ and R⁴ are H. In some embodiments, R⁵ is OH, H, or OSO₃H. In some embodiments, R⁵ is OH. In some embodiments, R⁶ is H, deuterium or CH₃. In some embodiments, R⁶ is H. In some embodiments, R⁷ is H or deuterium. In some embodiments, R⁷ is H. In some embodiments, X is NR⁸, R⁸ is ethyl, and R⁵ is OH.

In some embodiments, B is selected from the group consisting of —COR⁹, C₁-C₆ alkyl, and 5-6 membered heterocycloalkyl, wherein R⁹ is CH₃ or CD₃, the C₁-C₆ alkyl is further substituted with OH, D or both, and the 5-6 membered heterocycloalkyl is optionally substituted with CH₃. In some embodiments, B is C₁-C₆ alkyl is further substituted with OH. In some embodiments, B is C₁-C₆ alkyl is further substituted with OH and D. In some embodiments, B is ethyl substituted with OH. In some embodiments, B is ethyl substituted with OH and D. In some embodiments, B is isopropyl substituted with OH. In some embodiments, B is —COR⁹, wherein R⁹ is CH₃ or CD₃. In some embodiments, B is

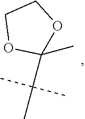

In some embodiments, C is H or deuterium. In some embodiments, C is H. In some embodiments, when X is CH, CD, or CH₂, then B is —COR⁹, wherein R⁹ is CH₃ or CD₃.

In some embodiments, the compound is selected from the group consisting of:

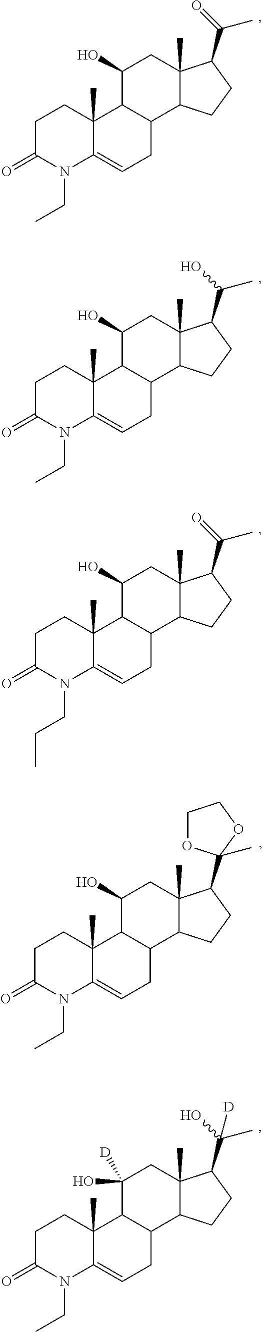 [SMILES: CCN1C(=O)CC[C@@]2(C)C1=CCC1C2[C@@H](O)C[C@@]2(C)C1CC[C@@H]2C(C)=O ; CCN1C(=O)CC[C@@]2(C)C1=CCC1C2[C@@H](O)C[C@@]2(C)C1CC[C@@H]2C(C)O ; CCCN1C(=O)CC[C@@]2(C)C1=CCC1C2[C@@H](O)C[C@@]2(C)C1CC[C@@H]2C(C)=O ; CCN1C(=O)CC[C@@]2(C)C1=CCC1C2[C@@H](O)C[C@@]2(C)C1CC[C@@H]2C1(C)OCCO1 ; [2H]C(C)(O)[C@H]1CCC2C3CC=C4N(CC)C(=O)CC[C@]4(C)C3[C@@]([2H])(O)C[C@@]21C]

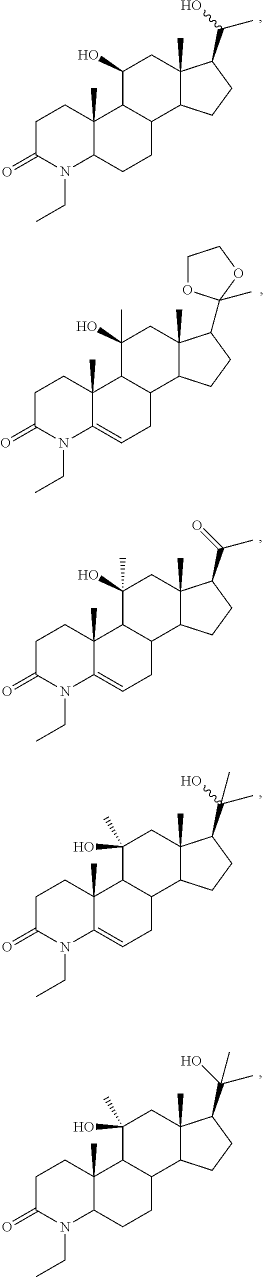 [SMILES: CCN1C(=O)CC[C@]2(C)C3C(CCC12)C1CC[C@H](C(C)O)[C@@]1(C)C[C@@H]3O ; CCN1C(=O)CC[C@@]2(C)C1=CCC1C3CCC(C4(C)OCCO4)[C@@]3(C)C[C@](C)(O)C12 ; CCN1C(=O)CC[C@@]2(C)C1=CCC1C2[C@@](C)(O)C[C@@]2(C)C1CC[C@@H]2C(C)=O ; CCN1C(=O)CC[C@@]2(C)C1=CCC1C2[C@@](C)(O)C[C@@]2(C)C1CC[C@@H]2C(C)(C)O ; CCN1C(=O)CC[C@@]2(C)C1CCC1C2[C@@](C)(O)C[C@@]2(C)C1CC[C@@H]2C(C)(C)O]

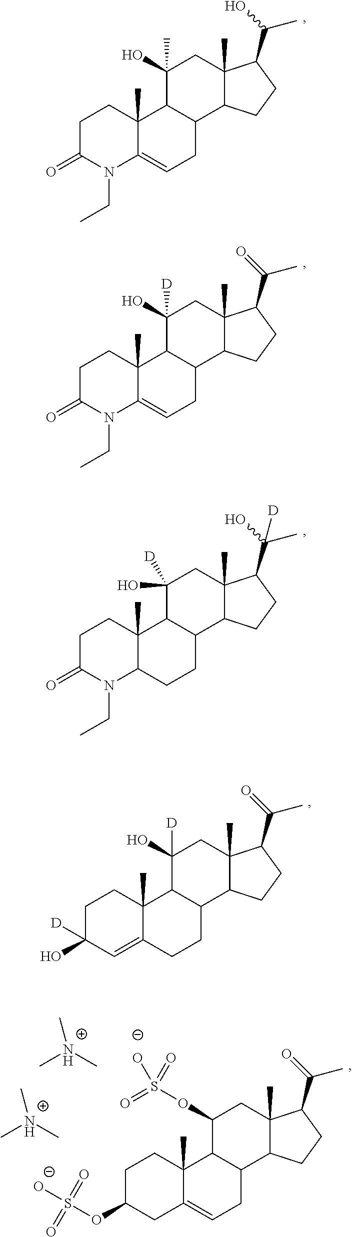

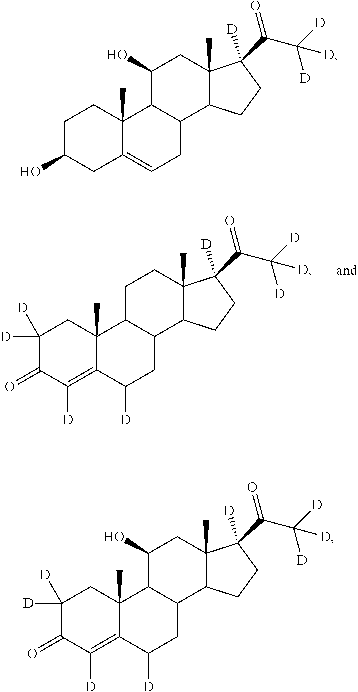

or a pharmaceutically acceptable salt, stereoisomer or tautomer thereof.

In some embodiments, the compound is not selected from the group consisting of 1-((3S,8S,9S,10R,11S,13S,14S,17S)-3,11-dihydroxy-10,13-dimethyl-2,3,4,7,8,9,10,11,12,13,14,15,16,17-tetradecahydro-1H-cyclopenta[a]phenanthren-17-yl)ethanone, 1-((3S,8S,9S,10R,11S,13S,14S,17S)-3,11-dihydroxy-10,13-dimethyl-2,3,6,7,8,9,10,11,12,13,14,15,16,17-tetradecahydro-1H-cyclopenta[a]phenanthren-17-yl)ethanone, (8S,9S,10R,11S,13S,14S,17S)-17-acetyl-11-hydroxy-10,13-dimethyl-6,7,8,9,10,11,12,13,14,15,16,17-dodecahydro-1H-cyclopenta[a]phenanthren-3(2H)-one, 1-((3S,8S,9S,10S,11S,13S,14S,17S)-3,11-dihydroxy-10,13-dimethylhexadecahydro-1H-cyclopenta[a]phenanthren-17-yl)ethan-1-one, 1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl)ethan1-one, (10R,11S,13S,17S)-17-acetyl-11-hydroxy-10,13-dimethyl-1,2,6,7,8,9,10,11,12,13,14,15,16,17-tetradecahydro-3H-cyclopenta[a]phenanthren-3-one, (10S,11S,13S,17S)-17-acetyl-11-hydroxy-10,13-dimethylhexadecahydro-3H-cyclopenta[a]phenanthren-3-one, (10R,11S,13S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one, 1-((3S,10S,11S,13S,17S)-3,11-dihydroxy-10,13-dimethylhexadecahydro-1H-cyclopenta[a]phenanthren-17-yl)ethan-1-one, 1-((3S,10R,11S,13 S,17S)-3,11-dihydroxy-10,13-dimethyl-2,3,4,7,8,9,10,11,12,13,14,15,16,17-tetradecahydro-1H-cyclopenta[a]phenanthren-17-yl-3-d)ethan-1-one, 1-((3S,10R,11S,13 S,17S)-3,11-dihydroxy-10,11,13-trimethyl-2,3,4,7,8,9,10,11,12,13,14,15,16,17-tetradecahydro-1H-cyclopenta[a]phenanthren-17-yl)ethan-1-one, 1-((3S,10R,11S,13 S,17S)-3,11-dihydroxy-10,13-dimethyl-2,3,4,7,8,9,10,11,12,13,14,15,16,17-tetradecahydro-1H-cyclopenta[a]phenanthren-17-yl-11-d)ethan-1-one, (8S,9S,10R,11S,13S,14S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one, 1-((3S,10R,11S,13 S,17S)-3,11-dihydroxy-10,13-dimethyl-2,3,4,7,8,9,10,11,12,13,14,15,16,17-tetradecahydro-1H-cyclopenta[a]phenanthren-17-yl-3,11-d₂)ethan-1-one, 1-((8S,9S,10R,11S,13 S,14S)-3,11-dihydroxy-10,13-dimethyl-2,3,4,7,8,9,10,11,12,13,14,15,16,17-tetradecahydro-1H-cyclopenta[a]phenanthren-17-yl)ethanone, (8S,9S,10R,11S,13 S,14S)-17-acetyl-1-hydroxy-10,13-dimethyl-6,7,8,9,10,11,12,13,14,15,16,17-dodecahydro-1H-cyclopenta[a]phenanthren-3(2H)-one, (3S,10S,11S,13S,17S)-17-((R)-1-hydroxyethyl)-10,13-dimethylhexadecahydro-1H-cyclopenta[a]phenanthrene-3,11-diol, (3S,10R,11S,13 S,17S)-17-((R)-1-hydroxyethyl)-10,13-dimethyl-2,3,6,7,8,9,10,11,12,13,14,15,16,17-tetradecahydro-1H-cyclopenta[a]phenanthrene-3,11-diol, (10R,11S,13S,17S)-11-hydroxy-17-((R)-1-hydroxyethyl)-10,13-dimethyl-1,2,6,7,8,9,10,11,12,13,14,15,16,17-tetradecahydro-3H-cyclopenta[a]phenanthren-3-one, (10R,11S,13 S,17S)-11-hydroxy-17-(2-hydroxypropan-2-yl)-10,13-dimethyl-1,2,6,7,8,9,10,11,12,13,14,15,16,17-tetradecahydro-3H-cyclopenta[a]phenanthren-3-one, (10R,11S,13 S,17S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one, (3S,10R,11S,13 S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthrene-3,11-diol, (3S,10R,11S,13 S,17S)-17-((R)-1-hydroxyethyl)-10,13-dimethyl-2,3,4,7,8,9,10,11,12,13,14,15,16,17-tetradecahydro-1H-cyclopenta[a]phenanthrene-3,11-diol, (3S,10R,11S,13R,17S)-17-ethyl-10,13-dimethyl-2,3,4,7,8,9,10,11,12,13,14,15,16,17-tetradecahydro-1H-cyclopenta[a]phenanthrene-3,11-diol, and trimethylammonium (3S,8S, 9S, 10R,11S,13S, 17S)-17-acetyl-11-hydroxy-10,13-dimethyl-2,3,4,7,8,9,10,11,12,13,14,15,16,17-tetradecahydro-1H-cyclopenta[a]phenanthren-3-yl sulfate, or a pharmaceutically acceptable salt, stereoisomer or tautomer thereof.

Representative compounds are listed in Table 1.

TABLE 1

| Compound | Name |
|---|---|
| 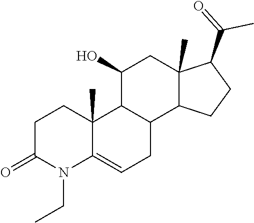 [SMILES: CCN1C(=O)CC[C@@]2(C)C1=CCC1C2[C@@H](O)C[C@@]2(C)C1CC[C@@H]2C(C)=O] | (4aR,5S,6aS,7S)-7-acetyl-1- ethyl-5-hydroxy-4a,6a-dimethyl- 1,3,4,4a,4b,5,6,6a,7,8,9,9a,9b,10- tetradecahydro-2H-indeno[5,4-f]- quinolin-2-one |
| 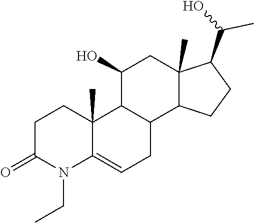 [SMILES: CCN1C(=O)CC[C@@]2(C)C1=CCC1C2[C@@H](O)C[C@@]2(C)C1CC[C@@H]2C(C)O] | (4aR,5S,6aS,7S)-1-ethyl-5- hydroxy-7-((R)-1-hydroxy- ethyl)-4a,6a-dimethyl- 1,3,4,4a,4b,5,6,6a,7,8,9,9a,9b,10- tetradecahydro-2H-indeno- [5,4-f]quinolin-2-one |
| 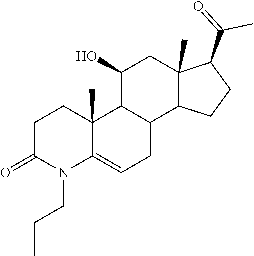 [SMILES: CCCN1C(=O)CC[C@@]2(C)C1=CCC1C2[C@@H](O)C[C@@]2(C)C1CC[C@@H]2C(C)=O] | (4aR,5S,6aS,7S)-7-acetyl-5- hydroxy-4a,6a-dimethyl-1-propyl- 1,3,4,4a,4b,5,6,6a,7,8,9,9a,9b,10- tetradecahydro-2H-indeno[5,4-f]- quinolin-2-one |
| 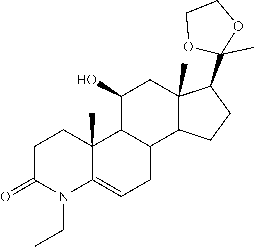 [SMILES: CCN1C(=O)CC[C@@]2(C)C1=CCC1C2[C@@H](O)C[C@@]2(C)C1CC[C@@H]2C1(C)OCCO1] | (4aR,5S,6aS,7S)-1-ethyl-5- hydroxy-4a,6a-dimethyl-7-(2- methyl-1,3-dioxolan-2-yl)- 1,3,4,4a,4b,5,6,6a,7,8,9,9a,9b,10- tetradecahydro-2H-indeno[5,4-f]- quinolin-2-one |
| 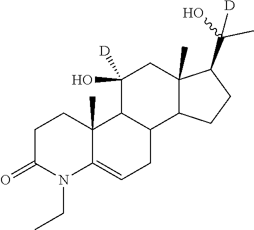 [SMILES: [2H]C(C)(O)[C@H]1CCC2C3CC=C4N(CC)C(=O)CC[C@]4(C)C3[C@@]([2H])(O)C[C@@]21C] | (4aR,5S,6aS,7S)-1-ethyl-5- hydroxy-7-(1-hydroxyethyl- 1-d)-4a,6a-dimethyl- 1,3,4,4a,4b,5,6,6a,7,8,9,9a,9b,10- tetradecahydro-2H-indeno- [5,4-f]quinolin-2-one-5-d |
| 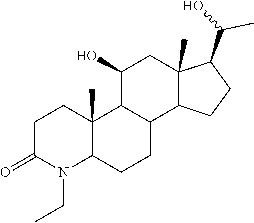 [SMILES: CCN1C(=O)CC[C@]2(C)C3C(CCC12)C1CC[C@H](C(C)O)[C@@]1(C)C[C@@H]3O] | (4aR,5S,6aS,7S)-1-ethyl-5- hydroxy-7-((R)-1-hydroxyethyl)- 4a,6a-dimethylhexadecahydro- 2H-indeno[5,4-f]quinolin-2-one |
| 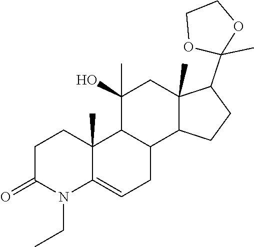 [SMILES: CCN1C(=O)CC[C@@]2(C)C1=CCC1C3CCC(C4(C)OCCO4)[C@@]3(C)C[C@](C)(O)C12] | (4aR,5S,6aS)-1-ethyl-5- hydroxy-4a,5,6a-trimethyl-7-(2- methyl-1,3-dioxolan-2-yl)- 1,3,4,4a,4b,5,6,6a,7,8,9,9a,9b,10- tetradecahydro-2H-indeno[5,4- f]quinolin-2-one |
| 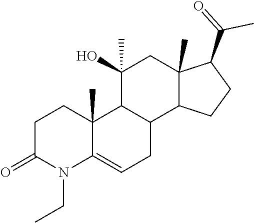 [SMILES: CCN1C(=O)CC[C@@]2(C)C1=CCC1C2[C@@](C)(O)C[C@@]2(C)C1CC[C@@H]2C(C)=O] | (4aR,5S,6aS,7S)-7-acetyl-1-ethyl- 5-hydroxy-4a,5,6a-trimethyl- 1,3,4,4a,4b,5,6,6a,7,8,9,9a,9b,10- tetradecahydro-2H-indeno[5,4-f]- quinolin-2-one |
| 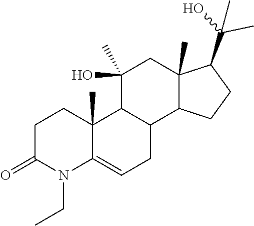 [SMILES: CCN1C(=O)CC[C@@]2(C)C1=CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan- 2-yl)-4a,5,6a-trimethyl- 1,3,4,4a,4b,5,6,6a,7,8,9,9a,9b,10- tetradecahydro-2H-indeno- [5,4-f]quinolin-2-one |
| 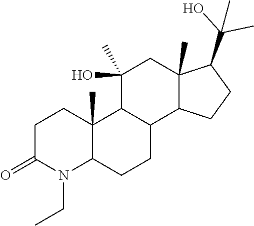 [SMILES: CCN1C(=O)CC[C@@]2(C)C1CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan-2- yl)-4a,5,6a-trimethylhexadeca- hydro-2H-indeno[5,4-f]quinolin- 2-one |
| 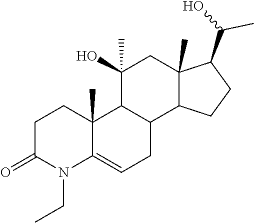 [SMILES: CCN1C(=O)CC[C@@]2(C)C1=CCC1C2[C@@](C)(O)C[C@@]2(C)C1CC[C@@H]2C(C)O] | (4aR,5S,6aS,7S)-1-ethyl-5- hydroxy-7-((R)-1-hydroxy- ethyl)-4a,5,6a-trimethyl- 1,3,4,4a,4b,5,6,6a,7,8,9,9a,9b,10- tetradecahydro-2H-indeno[5,4- f]quinolin-2-one |
| 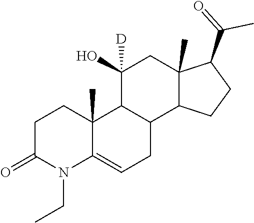 [SMILES: [2H][C@]1(O)C[C@@]2(C)C(CC[C@@H]2C(C)=O)C2CC=C3N(CC)C(=O)CC[C@]3(C)C21] | (4aR,5S,6aS,7S)-7-acetyl-1-ethyl- 5-hydroxy-4a,6a-dimethyl- 1,3,4,4a,4b,5,6,6a,7,8,9,9a,9b,10- tetradecahydro-2H-indeno[5,4- f]quinolin-2-one-5-d |
| 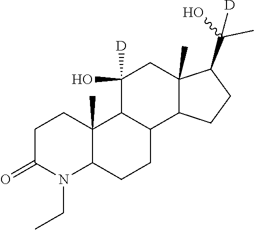 [SMILES: [2H]C(C)(O)[C@H]1CCC2C3CCC4N(CC)C(=O)CC[C@]4(C)C3[C@@]([2H])(O)C[C@@]21C] | (4aR,5S,6aS,7S)-1-ethyl-5- hydroxy-7-(1-hydroxyethyl-1-d)- 4a,6a-dimethylhexadecahydro- 2H-indeno[5,4-f]quinolin-2- one-5-d |
| 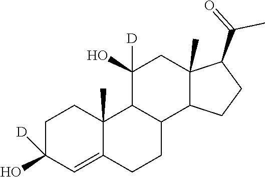 [SMILES: [2H][C@]1(O)C[C@@]2(C)C(CC[C@@H]2C(C)=O)C2CCC3=C[C@@]([2H])(O)CC[C@]3(C)C21] | 1-((3S,10R,11S,13S,17S)-3,11- dihydroxy-10,13-dimethyl- 2,3,6,7,8,9,10,11,12,13,14,15,16,17- tetradecahydro-1H-cyclopenta- [a]phenanthren-17-yl-3,11- d2)ethan-1-one |
| 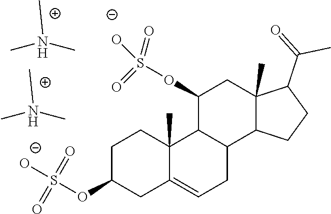 | (3S,10R,11S,13S)-17- acetyl-10,13-dimethyl- 2,3,4,7,8,9,10,11,12,13,14,15,16,17- tetradecahydro-1H-cyclopenta- [a]phenanthrene-3,11-diyl bis- (sulfate)trimethylammonium salt |
| 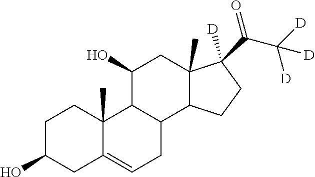 [SMILES: [2H]C([2H])([2H])C(=O)[C@@]1([2H])CCC2C3CC=C4C[C@@H](O)CC[C@]4(C)C3[C@@H](O)C[C@@]21C] | 1-((3S,10R,11S,13S,17S)-3,11- dihydroxy-10,13-dimethyl- 2,3,4,7,8,9,10,11,12,13,14,15,16,17- tetradecahydro-1H-cyclopenta[a]- phenanthren-17-yl-17-d)ethan-1- one-2,2,2-d3 |
| 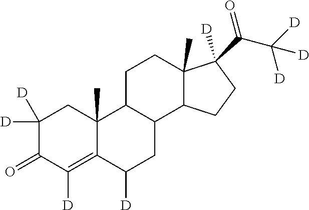 [SMILES: [2H]C1=C2C([2H])CC3C(CC[C@@]4(C)C3CC[C@]4([2H])C(=O)C([2H])([2H])[2H])[C@@]2(C)CC([2H])([2H])C1=O] | (10R,13S,17S)-17-(acetyl- d3)-10,13-dimethyl- 1,2,6,7,8,9,10,11,12,13,14,15,16,17- tetradecahydro-3H-cyclo- penta[a]phenanthren-3-one- 2,2,4,6,17-d5 |
| 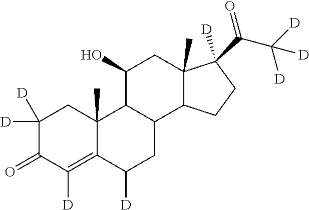 [SMILES: [2H]C1=C2C([2H])CC3C([C@@H](O)C[C@@]4(C)C3CC[C@]4([2H])C(=O)C([2H])([2H])[2H])[C@@]2(C)CC([2H])([2H])C1=O] | (10R,11S,13S,17S)-17-(acetyl- d3)-11-hydroxy-10,13-dimethyl- 1,2,6,7,8,9,10,11,12,13,14,15,16,17- tetradecahydro-3H-cyclopenta[a]- phenanthren-3-one-2,2,4,6,17-d5 |

Provided herein is a compound selected from the group consisting of the compounds described in Table 1.

Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

Compositions of any of the compounds detailed herein are embraced by this disclosure. In some embodiments, the present invention includes pharmaceutical compositions comprising a compound as detailed herein or pharmaceutically acceptable salt, stereoisomer or tautomer thereof and a pharmaceutically acceptable carrier or excipient.

Compounds described herein have 5′ AMP-activated protein kinase (AMPK) activities and can be useful in AMPK activation. In some embodiments, the present invention includes a method of preventing and/or treating such a disease or disorder in a subject in need thereof comprising administering to the subject a therapeutically effective amount of a compound as detailed herein, including but not limited to a compound of formula (II), a pharmaceutically acceptable salt, stereoisomer or tautomer thereof, or a pharmaceutical composition comprising such compound or salt.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to X of Formula (II) may be combined with every description, variation, embodiment or aspect of R¹, R², R³, R⁴, R⁵, R⁶, R⁷, B, and/or C the same as if each and every combination were specifically and individually listed.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity. In some embodiments, the provided compounds are sterilized.

The compounds described here also intend isotopically-labeled and/or isotopically-enriched forms. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into the provided compounds include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) is useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds can generally be prepared by standard methods and techniques known to those skilled in the art or by procedures similar to those described in the accompanying Examples substituting appropriate isotopically-labeled reagents in place of the corresponding non-labeled reagent.

### Methods of Preparation

The provided compounds may be prepared by a number of processes, including but not limited to the processes generally described below. In the following process descriptions, the symbols when used in the formulae depicted are to be understood to represent those groups described above in relation to the formulae herein.

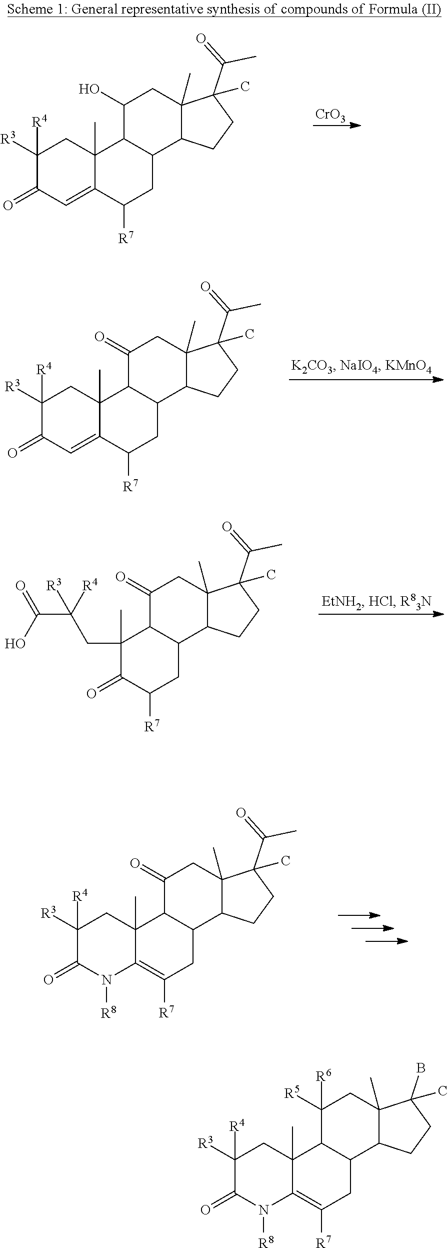

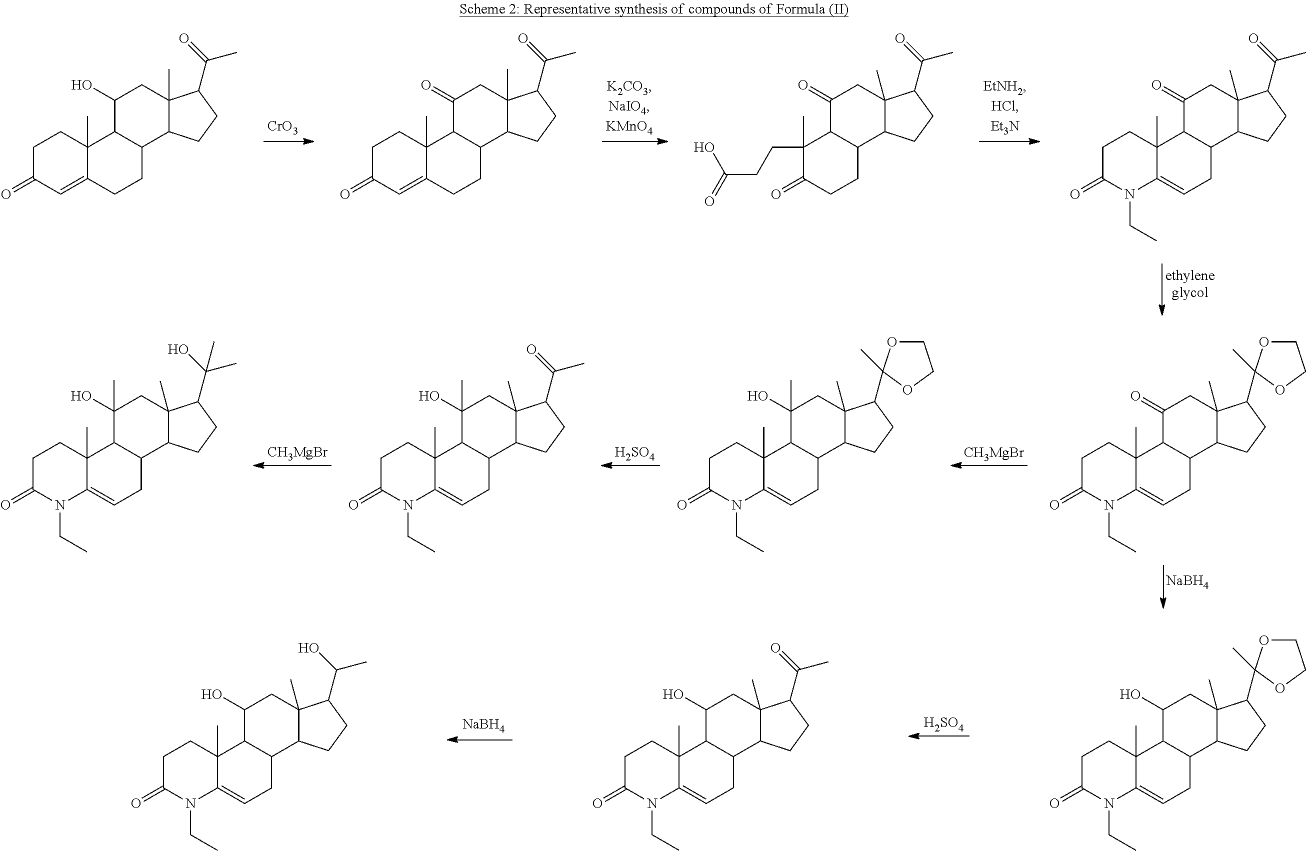

### Methods of Use

In certain embodiments, disclosed herein is a method of treatment of a mitochondrial biogenesis-mediated disease comprising the administration of a therapeutically effective amount of a compound as disclosed herein to a patient in need thereof.

In some embodiments, the mitochondrial biogenesis-mediated disease is selected from the group consisting of skeletal or cardiac muscle diseases associated with ischemia, or impaired or inadequate blood flow, diseases associated with genetic disorders that directly or indirectly affect the number, structure, or function of mitochondria, diseases associated with impaired neurological function associated with decreased mitochondrial number or function, diseases associated with loss of number, loss of function, or loss of correct, optimally efficient internal organization of skeletal muscle cells or cardiac muscle cells, metabolic diseases, and conditions associated with liver cell injury and altered fatty acid metabolism.

In some embodiments, the mitochondrial biogenesis-mediated disease is selected from the group consisting of acute coronary syndrome, myocardial infarction, angina, renal injury, renal ischemia, diseases of the aorta and its branches, injuries arising from medical interventions, atherosclerosis, trauma, diabetes, hyperlipidemia, vascular stenosis, peripheral arterial disease, vasculopathy, and vasculitis.

In some embodiments, the mitochondrial biogenesis-mediated disease is selected from the group consisting of Friedreich's ataxia, muscular dystrophy, Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, spinal muscular atrophy, and Emery-Dreifuss muscular dystrophy.

In some embodiments, the mitochondrial biogenesis-mediated disease is selected from the group consisting of Huntington's disease, Parkinson's disease, Alzheimer's disease, and amyotrophic lateral sclerosis.

In some embodiments, the mitochondrial biogenesis-mediated disease is sarcopenia.

In some embodiments, the mitochondrial biogenesis-mediated disease is selected from the group consisting of congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment.

In some embodiments, the mitochondrial biogenesis-mediated disease is selected from the group consisting of hepatic steatosis, hepatic fibrosis, cirrhosis, and hepatocyte or stellate cell injury.

In further embodiments, said method further comprises the administration of another therapeutic agent.

In some embodiments, the said agent is selected from the group consisting of hormones which stimulate muscle cell growth, γ-amino butyric acid or its derivatives, dietary protein supplements, anabolic steroids, biological factors known to enhance the growth, strength, endurance, or metabolism of skeletal or cardiac muscle, or recovery of skeletal muscle or cardiac muscle from injury or weakness, compounds known to be associated with increased nitric oxide production which promotes blood flow through muscles, extracts of natural products known to promote muscle strength or endurance, inhibitors of myostatin, stimulators of folistatin expression, compounds known to promote or facilitate mitochochondrial function or biogenesis, a tetracycline antibiotic, glycoprotein IIb/IIIa inhibitor, ADP receptor/P2Y12 inhibitor, prostaglandin analog, COX inhibitor, antiplatelet drug, anticoagulant, heparin, direct factor Xa inhibitor, direct thrombin (II) inhibitor, vasodilator.

In some embodiments, the said agent is doxycycline.

In some embodiments, the said agent is niacin or allopurinol.

In certain embodiments, disclosed herein are methods of treating or preventing the adverse effects of administration of compounds which exhibit mitochondrial toxicity comprising the administration of a therapeutically effective amount of a compound as disclosed herein to a patient in need thereof.

In some embodiments, the adverse effect is selected from the group consisting of abnormal mitochondrial respiration, abnormal oxygen consumption, abnormal extracellular acidification rate, abnormal mitochondrial number, abnormal lactate accumulation, and abnormal ATP levels.

In certain embodiments, disclosed herein are methods of improving muscle structure or function; improving mitochondrial effects associated with exercise; enhancing the capacity for exercise in those limited by age, inactivity, diet, or diseases; enhancing muscle health and function in response to exercise; enhancing muscle health and function in the clinical setting of restricted capacity for exercise; enhancing recovery of muscles from vigorous activity or from injury associated with vigorous or sustained activity, comprising the administration of a therapeutically effective amount of a compound as disclosed herein to a patient in need thereof.

In certain embodiments, disclosed herein are methods of enhancing sports performance and endurance, building muscle shape and strength, or facilitating recovery from the muscle related side effects of training or competition comprising the administration of a therapeutically effective amount of a compound as disclosed herein to a patient in need thereof.

In certain embodiments, disclosed herein are methods of stimulating increased number or function of skeletal muscle cells or contractile muscle cells comprising the administration of a therapeutically effective amount of a compound as disclosed herein to a patient in need thereof.

In some embodiments, the said stimulation of muscle cells comprises stimulation of cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes.

In certain embodiments, disclosed herein is a method of modulation of mitochondrial biogenesis comprising contacting mitochondria with a compound as disclosed herein.

In some embodiments, the mitochondrial biogenesis-mediated diseases include sarcopenia, muscular dystrophy, neurodegenerative diseases, liver disease, acute or chronic kidney failure, congestive heart failure, chronic obstructive pulmonary disorder (COPD), peripheral vascular disease, pulmonary hypertension, hyperlipidemia, hypertension, and diabetes.

In some embodiments, provided is a method of administering a compound or composition disclosed herein in an amount effective to stimulate the function, recovery, or regeneration of mitochondria or mitochondrial proteins or function. In further embodiments, provided is methods for preventing or treating adverse events or diseases associated with impaired mitochondrial number or function.

The term “optionally substituted” means the anteceding group may be substituted or unsubstituted.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “muscular diseases” refers to diseases associated with impaired skeletal muscle or cardiac muscle cell number or function.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein. In certain embodiments, a combination of compounds is administered such that the clearance half-life of each compound from the body overlaps at least partially with one another. For example, a first pharmaceutical has a clearance half-life of 1 hour and is administered at time=0, and a second pharmaceutical has a clearance half-life of 1 hour and is administered at time=45 minutes.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “pregnenolone and other related steroids” as used herein refers to any compound which retains the ring structure and the 11-oxo moiety of 11-hydroxy-pregnenolone itself, but which contains one or more substituent groups relative to 11-oxo-pregenolone. The term also includes prodrugs which release 11-hydroxy-pregnenolone when administered to a subject. The term also includes active metabolites of 11β-hydroxypregnenolone such as 11β-hydroxyprogesterone.

The term “11β-hydroxypregnenolone” as used herein refers to a compound having the structural formula

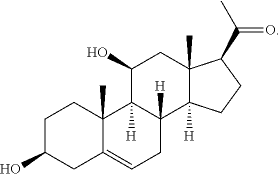

The term “11β-hydroxyprogesterone” as used herein refers to a compound having the structural formula:

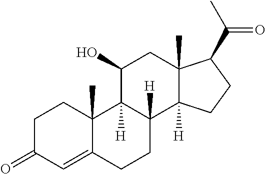

The term “derivative” as used herein to modify the term “11(3-hydroxypregnenolone” or “11β-hydroxysreoid” the term “11β-hydroxyprogesterone” refers to any compound which retains the ring structure and stereochemistry of 11β-hydroxypregnenolone or 11β-hydroxyprogesterone, “11β-hydroxysreoid” itself, but which contains one or more substituent groups relative to 11β-hydroxypregnenolone or 11β-hydroxyprogesterone. The term also includes combination molecules or prodrugs that release 11β-hydroxypregnenolone when administered to a subject. Such a combination molecule may include, for example, 11β-hydroxypregnenolone and an agent joined by a hydrolysable linker group.

The term “HCAEC” as used herein refer to Human corniary Artery endothelial cells; the term “BCAEC” refers to bovine corniary Artery endothelial cells; and the term “GAPDH” refers to glyceraldehyde-3-phosphate dehydrogenase.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. Included are compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, sodium, potassium, magnesium, and calcium salts of the compounds disclosed herein, and the like, are contemplated.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the provided compounds to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Specific sustained release formulations of the compounds disclosed herein are described in U.S. Pat. No. 6,410,052, which is hereby incorporated by reference.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. In an embodiment, the provided compounds may be used in a dosage in the range of 0.01 to 100 mg/Kg.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the provided compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds with agents which allow or enhance improvements in the number, structure or function of skeletal muscle cells or cardiac muscle cells.

Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of compounds or compositions disclosed herein in combination with agents which allow or enhance improvements in the number, structure, or function of skeletal muscle cells or cardiac muscle cells, cofactors that enhance mitochondrial biogenesis, and factors that enhance the production of NO in response to the stimulation of eNOS or nNOS.

In further embodiments, such agents include hormones which stimulate muscle cell growth, γ-amino butyric acid or its derivatives, dietary protein supplements, anabolic steroids, biological factors known to enhance the growth, strength, endurance, or metabolism of skeletal or cardiac muscle, or recovery of skeletal muscle or cardiac muscle from injury or weakness, compounds known to be associated with increased nitric oxide production which promotes blood flow through muscles, extracts of natural products known to promote muscle strength or endurance, inhibitors of myostatin, and stimulators of folistatin expression.

In further embodiments, hormones which stimulate muscle cell growth include, but are not limited to, growth hormone, growth hormone analogs, growth hormone releasing peptides or analogs thereof, growth hormone secretagogues, growth hormone precursors, or other hormones such as somatatropin or mechano growth factor.

In further embodiments, γ-amino butyric acid derivatives include, but are not limited to, 4-amino-3-phenylbutyric acid and neurotransmitters that benefit muscles by modulating the pituitary gland.

In further embodiments, dietary protein supplements include, but are not limited to, casein, whey, soy, egg white, hemp, rice, and pea proteins, amino acids precursors or derivatives thereof with known attributes of potentiating muscle growth, such as leucine, valine, isovaline, beta alanine, glutamine, glutamine dipeptide, or glycocyamine.

In further embodiments anabolic steroids, include, but are not limited to, testosterone or related steroid compounds with muscle growth inducing properties, such as cyclostanazol or methadrostenol, prohomones or derivatives thereof, modulators of estrogen, and selective androgen receptor modulators (SARMS).

In further embodiments, biological factors known to enhance the growth, strength, endurance, or metabolism of skeletal or cardiac muscle, or recovery of skeletal muscle or cardiac muscle from injury or weakness, include, but are not limited to, alpha-lipoic acid, taurine, caffeine, magnesium, niacin, folic acid, ornithine, vitamin B6, B12, or D, aspartate, creatine and its diverse salts such creatine monohydrate, betaine, N-acetyl cysteine, beta-hydroxyl methyl butyrate, lecithin, choline, phospholipid mixtures, phosphatidyl serine, carnitine, L-carnitine, acetyl-L-carnitine, and glycine proprionyl-L-carnitine.

In an embodiment, the compounds are used for the activation AMPK.

In an embodiment, the compounds are used for the subsequent activation transcription factors such as PCG-1 alpha associated with mitochondrial biogenesis.

In further embodiments, the compounds are used for the activation mitochondrial biogenesis and functions.

In further embodiments, the compounds are used for treatment of diseases associated with mitochondrial depletion and/or dysfunctionselected, but not limited to, the group consisting of skeletal muscle diseases, cardiac muscle diseases associated with ischemia, or impaired or inadequate blood flow, diseases associated with genetic disorders that directly or indirectly affect the number, structure, or function of mitochondria, diseases associated with impaired neurological function associated with decreased mitochondrial number or function, diseases associated with loss of number, loss of function, or loss of correct, optimally efficient internal organization of skeletal muscle cells or cardiac muscle cells, metabolic diseases, and conditions associated with liver cell injury and altered fatty acid metabolism.

In further embodiments, the compounds are used for mitochondrial biogenesis-mediated disease is selected from the group consisting of acute coronary syndrome, myocardial infarction, angina, renal injury, renal ischemia, diseases of the aorta and its branches, injuries arising from medical interventions, atherosclerosis, trauma, diabetes, hyperlipidemia, vascular stenosis, peripheral arterial disease, vasculopathy, and vasculitis, Friedreich's ataxia, muscular dystrophy, Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, spinal muscular atrophy, Emery-Dreifuss muscular dystrophy, Huntington's disease, Parkinson's disease, Alzheimer's disease, and amyotrophic lateral sclerosis, sarcopenia, congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment, hepatic steatosis, hepatic fibrosis, cirrhosis, and hepatocyte or stellate cell injury.

In further embodiments, compounds known to be associated with increased nitric oxide production which promotes blood flow through muscles include, but are not limited to, arginine and citrulline.

In further embodiments, compounds known to promote or facilitate mitochochondrial function or biogenesis, include, but are not limited to, alpha lipoic acid, resveratrol, coenzyme Q10 and its derivatives, forskalin, metformin, acetyl-carnitine, alpha tocopherol, pyruvate, choline, B vitamins, niacin, and biotin.

In further embodiments, extracts of natural products known to promote muscle strength or endurance, include, but are not limited to, guarana, geranium robertianum, cirsium ologophyllum, Bauhinia purpureae, yohimbe, Bacopa monniera, beet powder, rhodiola, or tea extracts.

In further embodiments, inhibitors of myostatin are proteins, antibodies, peptides, or small molecules.

In further embodiments, stimulators of follistatin expression or function are proteins, peptides, or small molecules.

In further embodiments, compounds disclosed herein may be administered in combination with another agent or agents such as niacin, or inhibitors of xanthine oxidase, such as allopurinol.

In further embodiments, compounds disclosed herein may be administered orally or parenterally.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating muscular diseases in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of muscular diseases.

The compositions may also be formulated as nutraceutical compositions. The term “nutraceutical composition” as used herein refers to a food product, foodstuff, dietary supplement, nutritional supplement or a supplement composition for a food product or a foodstuff comprising exogenously added compounds as disclosed herein. Details on techniques for formulation and administration of such compositions may be found in Remington, The Science and Practice of Pharmacy 21^{st} Edition (Mack Publishing Co., Easton, Pa.) and Nielloud and Marti-Mestres, Pharmaceutical Emulsions and Suspensions: 2^{nd} Edition (Marcel Dekker, Inc, New York).

As used herein, the term “food product” refers to any food or feed suitable for consumption by humans or animals. The food product may be a prepared and packaged food (e.g., mayonnaise, salad dressing, bread, grain bar, beverage, etc.) or an animal feed (e.g., extruded and pelleted animal feed, coarse mixed feed or pet food composition). As used herein, the term foodstuff refers to any substance fit for human or animal consumption.

Food products or foodstuffs are for example beverages such as nonalcoholic and alcoholic drinks as well as liquid preparation to be added to drinking water and liquid food, non-alcoholic drinks are for instance soft drinks, sport drinks, fruit juices, such as orange juice, apple juice, and grapefruit juice, lemonades, teas, near-water drinks, milk and other dairy drinks such as for example yogurt drinks, and diet drinks. In another embodiment food products or foodstuffs refer to solid or semi-solid foods comprising a provided compound. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

Animal feed including pet food compositions advantageously include food intended to supply necessary dietary requirements, as well as treats (e.g., dog biscuits) or other food supplements. The animal feed comprising the composition may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

The term “dietary supplement” refers to a small amount of a compound for supplementation of a human or animal diet packaged in single or multiple dose units.

Dietary supplements do not generally provide significant amounts of calories but may contain other micronutrients (e.g., vitamins or minerals). The term food products or foodstuffs also includes functional foods and prepared food products pre-packaged for human consumption.

The term nutritional supplement refers to a composition comprising a dietary supplement in combination with a source of calories. In some embodiments, nutritional supplements are meal replacements or supplements (e.g., nutrient or energy bars or nutrient beverages or concentrates).

Dietary supplements may be delivered in any suitable format. In certain embodiments, dietary supplements are formulated for oral delivery. The ingredients of the dietary supplement are contained in acceptable excipients and/or carriers for oral consumption. The actual form of the carrier, and thus, the dietary supplement itself, is not critical. The carrier may be a liquid, gel, gelcap, capsule, powder, solid tablet (coated or noncoated), tea, or the like.

Compounds and compositions disclosed herein are administered in an “effective amount.” This term is defined hereinafter. Unless dictated otherwise, explicitly or otherwise, an “effective amount” is not limited to a minimal amount sufficient to ameliorate a condition, or to an amount that results in an optimal or a maximal amelioration of the condition. In the case when two or more compounds are administered together, an effective amount of one such compound may not be, in and of itself, be an effective amount, but may be an effective amount when used together with additional compounds.

In certain embodiments, the effective amount is an amount which stimulates mitochondrial function in cells. Such stimulation of mitochondrial function in cells may comprise stimulation of one or more of mitochondrial respiration and mitochondrial biogenesis. The methods and compositions described herein can assist in prevention of the consequences of mitochondrial toxicity which has not yet occurred, as well as provide for the active therapy of mitochondrial toxicity that may have already occurred.

While the phrase “administered together” as used herein may refer to the provision of chemical compositions in the same pharmaceutical composition, the phrase as used herein is not intended to imply that this must be so. Rather, two or more chemical compositions are “administered together” if the T_{1/2} for the clearances of each composition from the body overlaps at least partially with one another. For example, if a first pharmaceutical has a T_{1/2} for clearance of 1 hour and is administered at time=0, and a second pharmaceutical has a T_{1/2} for clearance of 1 hour and is administered at time=45 minutes, such pharmaceuticals are considered administered together. Conversely, if the second drug is administered at time=2 hours, such pharmaceuticals are not considered administered together.

Routes of administration for the pharmaceutical compositions include parenteral and enteral routes. Enteral routes of administration include delivery by mouth (oral), nasal, rectal, and vaginal routes. Parenteral routes of administration include intravenous, intramuscular, subcutaneous, and intraperitoneal routes. When more than one pharmaceutical composition is being administered, each need not be administered by the same route. In particularly embodiments, 11β-hydroxypregnenolone, or a derivative or pharmaceutically acceptable salt thereof, is administered together intravenously with one or more tetracycline antibiotics such as doxycycline, most preferably in a single pharmaceutical composition.

In certain embodiments, the methods disclosed herein comprise the administration to cells at least 10 pM, at least 1.0 nM, or at least 100 nM of a compounds disclosed herein.

In further embodiments, the methods disclosed herein comprise the administration of compounds of the disclosure in a total daily dose of about 0.001 mg/kg/dose to about 10 mg/kg/dose, alternately from about 0.3 mg/kg/dose to about 3 mg/kg/dose. In another embodiment the dose range is from about 0.1 to about 1 mg/kg/day. Generally between about 0.01 mg and about 0.1 gram per day can be administered; alternately between about 2.5 mg and about 200 mg can be administered. The dose may be administered in as many divided doses as is convenient.

In further embodiments, the methods disclosed herein comprise the administration of compounds disclosed herein in a range of about 0.1 to about 100 mg per kg body weight.

In further embodiments, the desired concentration is maintained for at least 30 minutes, 1 hour, 3 hours, 6 hours, 12 hours, 24 hours, 48 hours, 72 hours, or more. In yet further embodiments, the desired concentration is achieved at least once during each 12-hour period over at least 24 hours, 48 hours, 72 hours, 1 week, one month, or more; or at least once during each 24-hour period over at least 48 hours, 72 hours, 1 week, one month, or more. In order to maintain a desired concentration for a desired time, multiple doses of one or more compounds may be employed. The dosing interval may be determined based on the clearance half-life for each compound of interest from the body.

In certain embodiments, disclosed herein are methods and compositions for the treatment of diseases associated with loss of number, function, or correct, optimally efficient internal organization of mitochondria within cells.

In further embodiments, disclosed herein are methods and compositions for the treatment of skeletal or cardiac muscle diseases associated with ischemia, or impaired or inadequate blood flow. Examples of such states include, but are not limited to, atherosclerosis, trauma, diabetes, vascular stenosis, peripheral arterial disease, vasculopathy, and vasculitis.

In further embodiments, disclosed herein are methods and compositions for the treatment of diseases associated with genetic disorders that directly or indirectly affect the number, structure, or function of mitochondria, particularly those associated with muscle dysfunction or myopathy. Examples of such states include, but are not limited to, the set of diseases broadly classified as muscular dystrophies and Friedreich's ataxia. In further embodiments, said muscular dystrophy is selected from the group consisting of Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

In further embodiments, disclosed herein are methods and compositions for the therapeutic treatment of diseases associated with impaired neurological function associated with decreased mitochondrial number or function. Examples include, but are not limited to, Huntington's disease, Parkinson's disease, Alzheimer's disease, and amyotrophic lateral sclerosis (ALS).

In certain embodiments, disclosed herein are methods and compositions for the treatment of diseases associated with loss of number, loss of function, or loss of correct, optimally efficient internal organization of skeletal muscle cells or cardiac muscle cells. Such diseases may eventuate in a state of functionally significant muscle wasting, which, in its most pronounced form, is termed sarcopenia. Sarcopenia may be secondary to a variety of disorders, including aging, muscular dystrophy, diabetes, or other abnormal metabolic conditions, infection, inflammation, autoimmune disease, cardiac dysfunction, or severe disuse syndromes, or inactivity associated with arthritis. Examples of such diseases include, but are not limited to, congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment.

In certain embodiments, disclosed herein are methods and compositions for use to enhance sports performance and endurance, to build muscle shape and strength, and to facilitate recovery from the muscle related side effects of training or competition, such as soreness, weakness, cramping, pain, or injury.

In certain embodiments, a subject is selected for treatment with a compound or composition disclosed herein based on the occurrence of one or more physiological manifestations of skeletal or cardiac muscle injury or dysfunction in the subject. Such manifestations include elevations in biomarkers known to be related to injury of the heart or skeletal muscle. Examples of such biomarkers include, but are not limited to, elevated plasma levels of cardiac or skeletal muscle enzymes or proteins, such as myoglobin, troponin, or creatine phosphokinase, lactic acidosis, and elevated serum creatinine.

In certain embodiments, a compound or composition as disclosed herein is administered in an amount which stimulates increased number or function of skeletal muscle cells or contractile muscle cells. Such stimulation of muscle cells may comprise stimulation of one or more aspects of muscle cell function, including cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes. The methods and compositions described herein can assist in prevention of the consequences of muscle injury or dysfunction which have not yet occurred, as well as provide for the active therapy of muscle injury, dysfunction, or diseases which have already occurred.

In yet another aspect, provided is methods of treating metabolic disease in a subject. These methods comprise administering to a subject in need thereof a compound disclosed herein. In certain embodiments, the subject is selected based on the occurrence of diabetes or hyperlipidemia. In further embodiments the method reduces blood glucose levels and/or lowers blood triglycerides in the subject.

In yet another aspect, provided is methods of preventing or reversing injury to hepatic mitochondria by agents such as fructose, and thus preventing or reversing hepatic steatosis and other conditions associated with liver cell injury and altered fatty acid metabolism, and thus preventing or reversing hepatic fibrosis, or cirrhosis, associated with sustained hepatocyte or stellate cell injury.

In another embodiment, compounds or compositions disclosed herein may be administered in combination with another agent or agents such as niacin, or inhibitors of xanthine oxidase, such as allopurinol, to treat hyperlipidemia or treat liver injury associated with fructose or other agents associated with intracellular fat accumulation, steatosis, fibrosis, or cirrhosis.

In another embodiment, a decrease in the plasma or tissue levels of 11β-hydroxypregnenolone, 11β-hydroxyprogesterone, or metabolites thereof, such as sulfated, glucuronidated, or methylated derivatives, may be employed as a diagnostic test to determine deficiency states of 11β-hydroxypregnenolone or 11β-hydroxyprogesterone.

In another embodiment, an increase in the plasma or tissue levels of 11β-hydroxypregnenolone, 11β-hydroxyprogesterone, or metabolites thereof, such as sulfated, glucoronidated, or methylated derivatives, may be employed as a diagnostic test to determine therapeutic response to administration of 11β-hydroxypregnenolone, 11β-hydroxyprogesterone, or derivatives thereof.

Changes in the plasma or tissue levels of 11β-hydroxypregnenolone or 11β-hydroxyprogesterone or related hydroxysteroids and metabolites thereof may also be employed in conjunction with biomarkers of muscle injury or regeneration, such as myostatin, follistatin, creatine kinase, and others in order to determine deficiency states of the hydroxysteroid pathway or measure therapeutic response to hydroxysteroid-based therapeutics.

In another embodiment, compounds or compositions disclosed herein may be employed as therapeutics, or hormone replacement, in diseases or conditions associated with deficiency states of 11β-hydroxypregnenolone, 11β-hydroxyprogesterone, or metabolites thereof.

In a first aspect, provided is methods for preventing or treating adverse events associated with the use of chemical compositions such as approved medications in which the adverse event is caused by, or associated with, perturbations in mitochondrial number, function, or structure. The methods comprise administering to a subject in need thereof a compound or composition disclosed herein. In certain embodiments the method reduces symptoms of mitochondrial toxicity due to the subject's exposure to chemical compositions that exhibit mitochondrial toxicity.

In certain embodiments, compounds or compositions disclosed herein are administered in combination with one or more chemical compositions which exhibit mitochondrial toxicity. Such chemical compositions include, but are not limited to, those described above in regard to drug-induced mitochondrial dysfunction of the heart, liver, and kidneys.

In certain embodiments, the chemical composition that exhibits mitochondrial toxicity is identified based on the demonstration of one or more biological effects indicative of mitochondrial toxicity by the chemical composition. Such effects include, but are not limited to, abnormal mitochondrial respiration, abnormal oxygen consumption, abnormal extracellular acidification rate, abnormal mitochondrial number, abnormal lactate accumulation, abnormal ATP levels, etc.

In other embodiments, compounds or compositions disclosed herein are administered based on the occurrence of one or more physiological manifestations of mitochondrial toxicity in the subject. Such manifestations include, but are not limited to, elevations in markers known to relate to injury to the heart, liver, and/or kidney. Non-limiting examples include elevated serum liver enzymes, elevated cardiac enzymes, lactic acidosis, elevated blood glucose, elevated serum creatinine, etc.

In certain embodiments, compounds or compositions disclosed herein are administered in combination with one or more chemical compositions which can increase the biological activity of compounds disclosed herein, particularly with respect to effecting mitochondrial biogenesis, promoting muscle regeneration, and enhancing NO availability via the stimulation of the expression and activity of eNOS and nNOS.

In another embodiment, provided is methods for improving muscle structure or function; methods for improving mitochondrial effects associated with exercise; methods for enhancing the capacity for exercise in those limited by age, inactivity, diet, or any of the aforementioned diseases and conditions; methods for enhancing muscle health and function in response to exercise; methods for enhancing muscle health and function in the clinical setting of restricted capacity for exercise, whether due to injury, inactivity, obesity, or any of the aforementioned diseases and conditions; and/or methods to enhance recovery of muscles from vigorous activity or from injury associated with vigorous or sustained activity.

In related aspects, provided is methods of treating a condition involving decreased mitochondrial function in an animal. These methods comprise delivering to the animal one or more compounds or compositions disclosed herein.

In some embodiments, provided is compositions and methods for prophylactic and/or therapeutic treatment of diseases and conditions related to apoptosis and cellular necrosis caused by ischemia. In various aspects described hereinafter, provided is compositions and methods for treatment of acute coronary syndromes, including but not limited to myocardial infarction and angina; acute ischemic events in other organs and tissues, including but not limited to renal injury, renal ischemia and diseases of the aorta and its branches; injuries arising from medical interventions, including but not limited to coronary artery bypass grafting (CABG) procedures and aneurysm repair; and metabolic diseases, including but not limited to diabetes mellitus.

In some embodiments, provided is compositions and methods for prophylactic and/or therapeutic treatment of conditions related to mitochondrial function. In various aspects described hereinafter, provided is a method comprising administering one or more compounds as disclosed herein. Stimulation of mitochondrial function in cells may comprise stimulation of one or more of mitochondrial respiration and mitochondrial biogenesis. The methods and compositions described herein can assist in prevention of impaired mitochondria biogenesis and thus prevention of the consequences of impaired mitochondrial biogenesis in various diseases and conditions, as well as provide for the active therapy of mitochondrial depletion that may have already occurred.

In certain embodiments, compounds or compositions disclosed herein are administered to the subject together with one or more additional drugs useful in the treatment of ischemic or ischemia/reperfusion events. Exemplary additional drugs include one or more compounds independently selected from the group consisting of tetracycline antibiotics (e.g., doxycycline), glycoprotein Iib/IIIa inhibitors (e.g., eptifibatide, tirofiban, abciximab); ADP receptor/P2Y12 inhibitors (e.g., clopidogrel, ticlopidine, prasgurel); prostaglandin analogues (e.g., betaprost, iloprost, trepostinil); COX inhibitors (e.g., asprin, aloxiprin); other antiplatelet drugs (e.g., ditazole, cloricromen, dipyridamole, indobufen, picotamide, triflusal); anticoagulants (e.g., coumarins, 1,3-indandiones); heparins; direct factor Xa inhibitors; direct thrombin (II) inhibitors (e.g., bivalirudin); and vasodilators (e.g., fendoldopam, hydralazine, nesiritide, nicorandil, nicardipine, nitroglycerine, nitroprusside). This list is not meant to be limiting. In a particularly embodiment, a compound or composition disclosed herein is administered together with one or more tetracycline antibiotics such as doxycycline.

In the case of an ischemic event involving the heart, objective measures include increases in one or more cardiac markers (e.g., CK-MB, myoglobin, cardiac troponin I, cardiac troponin T, B-type Natriuretic peptide, NT-proBNP, etc.); changes in serial ECG tracings; and angiographic results.

In the case of an ischemic event involving the kidneys, objective measures include those defined by Bellomo et al., Crit Care. 8(4):R204-12, 2004, which is hereby incorporated by reference in its entirety. This reference proposes the following classifications for stratifying acute kidney injury patients: “Risk”: serum creatinine increased 1.5 fold from baseline OR urine production of <0.5 ml/kg body weight for 6 hours; “Injury”: serum creatinine increased 2.0 fold from baseline OR urine production <0.5 ml/kg for 12 h; “Failure”: serum creatinine increased 3.0 fold from baseline OR creatinine >355 μmol/L (with a rise of >44) or urine output below 0.3 ml/kg for 24 h.

In a related aspect, provided is pharmaceutical compositions for treatment of an acute ischemic or ischemia/reperfusion (IR) event. This composition comprises an effective amount of a compound or composition disclosed herein, and one or more additional drugs useful in the treatment of ischemic or ischemia/reperfusion events. In particularly embodiments, the pharmaceutical composition comprises a compound or composition disclosed herein and one or more tetracycline antibiotics, such as doxycycline. In a further embodiment, the composition is formulated for intravenous delivery.

### Kits

Also provided are articles of manufacture and kits containing any of the compounds and compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing the compounds and compositions described herein and instructions for use. The kits may contain instructions for use in the treatment of any disease provided herein in a subject in need thereof. A kit may additionally contain any materials or equipment that may be used in the administration of the compounds and compositions, such as vials, syringes, or IV bags. A kit may also contain sterile packaging.

### ENUMERATED EMBODIMENTS

1. A compound of Formula (II),

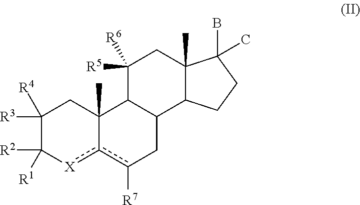

or a pharmaceutically acceptable salt, stereoisomer or tautomer thereof, wherein:

[symbol] is independently either a single bond or a double bond, provided that adjacent double bonds are not allowed;

R¹ is ═O, OH or OSO₃H

R² is absent, H or deuterium;

R³ and R⁴ are independently H or deuterium;

R⁵ is OH, H or OSO₃H

R⁶ is H, deuterium or CH₃;

R⁷ is H or deuterium;

X is CH, CD, CH₂ or NR⁸, wherein R⁸ is C₁-C₆ alkyl;

B is selected from the group consisting of —COR⁹, C₁-C₆ alkyl, and 5-6 membered heterocycloalkyl, wherein R⁹ is CH₃ or CD₃, the C₁-C₆ alkyl is further substituted with OH, D or both, and the 5-6 membered heterocycloalkyl is optionally substituted with CH₃; and

C is H or deuterium;

provided that when X is CH, CD, or CH₂, then B is —COR⁹, wherein R⁹ is CH₃ or CD₃, and the compound is not selected from the group consisting of:
1-((3S,8S,9S,10R,11S,13S,14S,17S)-3,11-dihydroxy-10,13-dimethyl-2,3,4,7,8,9,10,11,12,13,14,15,16,17-tetradecahydro-1H-cyclopenta[a]phenanthren-17-yl)ethanone,
1-((3S,8S,9S,10R,11S,13S,14S,17S)-3,11-dihydroxy-10,13-dimethyl-2,3,6,7,8,9,10,11,12,13,14,15,16,17-tetradecahydro-1H-cyclopenta[a]phenanthren-17-yl)ethanone,
(8S,9S,10R,11S,13S,14S,17S)-17-acetyl-11-hydroxy-10,13-dimethyl-6,7,8,9,10,11,12,13,14,15,16,17-dodecahydro-1H-cyclopenta[a]phenanthren-3(2H)-one,
1-((3S,8S,9S,10S,11S,13S,14S,17S)-3,11-dihydroxy-10,13-dimethylhexadecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl)ethan1-one,
(10R,11S,13S,17S)-17-acetyl-11-hydroxy-10,13-dimethyl-1,2,6,7,8,9,10,11,12,13,14,15,16,17-tetradecahydro-3H-cyclopenta[a]phenanthren-3-one,
(10S,11S,13S,17S)-17-acetyl-11-hydroxy-10,13-dimethylhexadecahydro-3H-cyclopenta[a]phenanthren-3-one,
(10R,11S,13S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10S,11S,13S,17S)-3,11-dihydroxy-10,13-dimethylhexadecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3-d)ethan-1-one,
1-((3S,10R,11S,13S,17S)-3,11-dihydroxy-10,11,13-trimethyl-2,3,4,7,8,9,10,11,12,13,14,15,16,17-tetradecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-11-d)ethan-1-one,
(8S,9S,10R,11S,13S,14S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3,11-d₂)ethan-1-one,
1-((8S,9S,10R,11S,13 S,14S)-3,11-dihydroxy-10,13-dimethyl-2,3,4,7,8,9,10,11,12,13,14,15,16,17-tetradecahydro-1H-cyclopenta[a]phenanthren-17-yl)ethanone,
(8S,9S,10R,11S,13S,14S)-17-acetyl-1-hydroxy-10,13-dimethyl-6,7,8,9,10,11,12,13,14,15,16,17-dodecahydro-1H-cyclopenta[a]phenanthren-3(2H)-one,
(3S,10S,11S,13S,17S)-17-((R)-1-hydroxyethyl)-10,13-dimethylhexadecahydro-1H-cyclopenta[a]phenanthrene-3,11-diol,
(3S,10R,11S,13S,17S)-17-((R)-1-hydroxyethyl)-10,13-dimethyl-2,3,6,7,8,9,10,11,12,13,14,15,16,17-tetradecahydro-1H-cyclopenta[a]phenanthrene-3,11-diol,
(10R,11S,13S,17S)-11-hydroxy-17-((R)-1-hydroxyethyl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxypropan-2-yl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one,
(3S,10R,11S,13S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthrene-3,11-diol,
(3S,10R,11S,13S,17S)-17-((R)-1-hydroxyethyl)-10,13-dimethyl-2,3,4,7,8,9,10,11,12,13,14,15,16,17-tetradecahydro-1H-cyclopenta[a]phenanthrene-3,11-diol,
(3S,10R,11S,13R,17S)-17-ethyl-10,13-dimethyl-2,3,4,7,8,9,10,11,12,13,14,15,16,17-tetradecahydro-1H-cyclopenta[a]phenanthrene-3,11-diol, and
Trimethylammonium (3S, 8S, 9S, 10R,11S,13S, 17S)-17-acetyl-11-hydroxy-10,13-dimethyl-2,3,4,7,8,9,10,11,12,13,14,15,16,17-tetradecahydro-1H-cyclopenta[a]phenanthren-3-yl sulfate,
   or a pharmaceutically acceptable salt, stereoisomer or tautomer thereof.
   2. The compound of embodiment 1, or a pharmaceutically acceptable salt, stereoisomer or tautomer thereof, wherein X is NR⁸, wherein R⁸ or C₁-C₆ alkyl.
   3. The compound of embodiment 1, or a pharmaceutically acceptable salt, stereoisomer or tautomer thereof, wherein X is CH, CD, or CH₂.
   4. The compound of any one of embodiments 1-3, or a pharmaceutically acceptable salt, stereoisomer or tautomer thereof, wherein R¹ is ═O and R² is absent.
   5. The compound of any one of embodiments 1-4, or a pharmaceutically acceptable salt, stereoisomer or tautomer thereof, wherein R³ and R⁴ are H.
   6. The compound of any one of embodiments 1-5, or a pharmaceutically acceptable salt, stereoisomer or tautomer thereof, wherein R⁵ is OH.
   7. The compound of any one of embodiments 1-6, or a pharmaceutically acceptable salt, stereoisomer or tautomer thereof, wherein R⁶ is H.
   8. The compound of any one of embodiments 1-7, or a pharmaceutically acceptable salt, stereoisomer or tautomer thereof, wherein B is —COR⁹, wherein R⁹ is CH₃ or CD₃.
   9. The compound of embodiment 1, or a pharmaceutically acceptable salt, stereoisomer or tautomer thereof, wherein the compound is of formula (II-a).

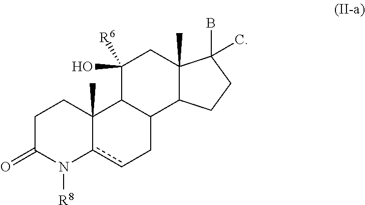

10. A compound, or a pharmaceutically acceptable salt, stereoisomer or tautomer thereof, wherein the compound is selected from the group consisting of:

| Compound | Name |
|---|---|
| 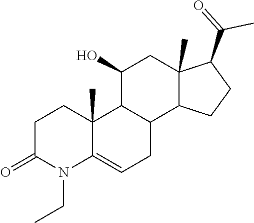 [SMILES: CCN1C(=O)CC[C@@]2(C)C1=CCC1C2[C@@H](O)C[C@@]2(C)C1CC[C@@H]2C(C)=O] | (4aR,5S,6aS,7S)-7-acetyl-1-ethyl- 5-hydroxy-4a,6a-dimethyl- 1,3,4,4a,4b,5,6,6a,7,8,9,9a,9b,10- tetradecahydro-2H-indeno[5,4- f]quinolin-2-one |
| 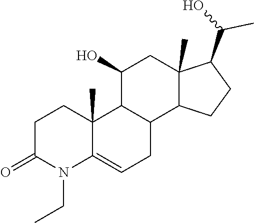 [SMILES: CCN1C(=O)CC[C@@]2(C)C1=CCC1C2[C@@H](O)C[C@@]2(C)C1CC[C@@H]2C(C)O] | (4aR,5S,6aS,7S)-1-ethyl- 5-hydroxy-7-((R)-1-hydroxy- ethyl)-4a,6a-dimethyl- 1,3,4,4a,4b,5,6,6a,7,8,9,9a,9b,10- tetradecahydro-2H-indeno- [5,4-f]quinolin-2-one |
| 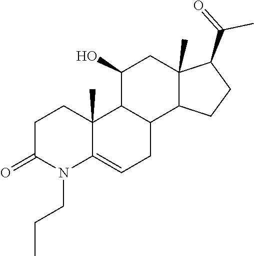 [SMILES: CCCN1C(=O)CC[C@@]2(C)C1=CCC1C2[C@@H](O)C[C@@]2(C)C1CC[C@@H]2C(C)=O] | (4aR,5S,6aS,7S)-7-acetyl-5- hydroxy-4a,6a-dimethyl-1-propyl- 1,3,4,4a,4b,5,6,6a,7,8,9,9a,9b,10- tetradecahydro-2H-indeno[5,4- f]quinolin-2-one |
| 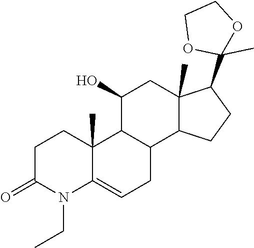 [SMILES: CCN1C(=O)CC[C@@]2(C)C1=CCC1C2[C@@H](O)C[C@@]2(C)C1CC[C@@H]2C1(C)OCCO1] | (4aR,5S,6aS,7S)-1-ethyl-5- hydroxy-4a,6a-dimethyl-7-(2- methyl-1,3-dioxolan-2-yl)- 1,3,4,4a,4b,5,6,6a,7,8,9,9a,9b,10- tetradecahydro-2H-indeno[5,4- f]quinolin-2-one |
| 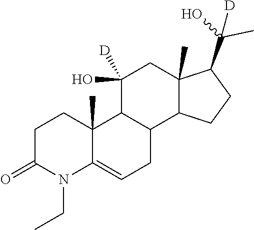 [SMILES: [2H]C(C)(O)[C@H]1CCC2C3CC=C4N(CC)C(=O)CC[C@]4(C)C3[C@@]([2H])(O)C[C@@]21C] | (4aR,5S,6aS,7S)-1-ethyl-5- hydroxy-7-(1-hydroxyethyl- 1-d)-4a,6a-dimethyl- 1,3,4,4a,4b,5,6,6a,7,8,9,9a,9b,10- tetradecahydro-2H-indeno[5,4- f]quinolin-2-one-5-d |
| 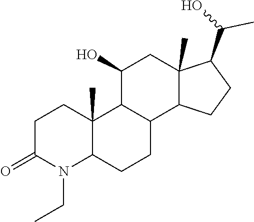 [SMILES: CCN1C(=O)CC[C@]2(C)C3C(CCC12)C1CC[C@H](C(C)O)[C@@]1(C)C[C@@H]3O] | (4aR,5S,6aS,7S)-1-ethyl-5- hydroxy-7-((R)-1-hydroxyethyl)- 4a,6a-dimethylhexadecahydro- 2H-indeno[5,4-f]quinolin-2-one |
| 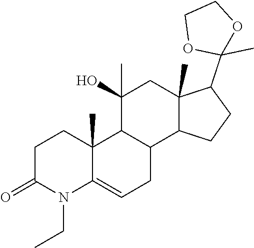 [SMILES: CCN1C(=O)CC[C@@]2(C)C1=CCC1C3CCC(C4(C)OCCO4)[C@@]3(C)C[C@](C)(O)C12] | (4aR,5S,6aS)-1-ethyl-5- hydroxy-4a,5,6a-trimethyl-7-(2- methyl-1,3-dioxolan-2-yl)- 1,3,4,4a,4b,5,6,6a,7,8,9,9a,9b,10- tetradecahydro-2H-indeno[5,4- f]quinolin-2-one |
| 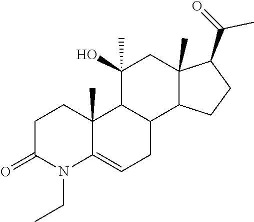 [SMILES: CCN1C(=O)CC[C@@]2(C)C1=CCC1C2[C@@](C)(O)C[C@@]2(C)C1CC[C@@H]2C(C)=O] | (4aR,5S,6aS,7S)-7-acetyl-1- ethyl-5-hydroxy-4a,5,6a-trimethyl- 1,3,4,4a,4b,5,6,6a,7,8,9,9a,9b,10- tetradecahydro-2H-indeno[5,4-f]- quinolin-2-one |
| 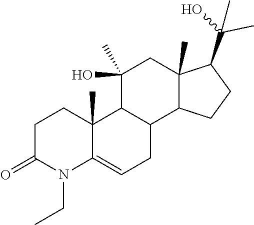 [SMILES: CCN1C(=O)CC[C@@]2(C)C1=CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan-2- yl)-4a,5,6a-trimethyl- 1,3,4,4a,4b,5,6,6a,7,8,9,9a,9b,10- tetradecahydro-2H-indeno[5,4-f]- quinolin-2-one |
| 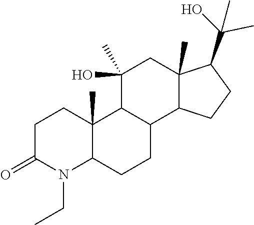 [SMILES: CCN1C(=O)CC[C@@]2(C)C1CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan-2- yl)-4a,5,6a-trimethylhexadeca- hydro-2H-indeno[5,4-f]quinolin- 2-one |
| 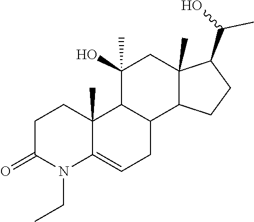 [SMILES: CCN1C(=O)CC[C@@]2(C)C1=CCC1C2[C@@](C)(O)C[C@@]2(C)C1CC[C@@H]2C(C)O] | (4aR,5S,6aS,7S)-1-ethyl-5- hydroxy-7-((R)-1-hydroxy- ethyl)-4a,5,6a-trimethyl- 1,3,4,4a,4b,5,6,6a,7,8,9,9a,9b,10- tetradecahydro-2H-indeno- [5,4-f]quinolin-2-one |
| 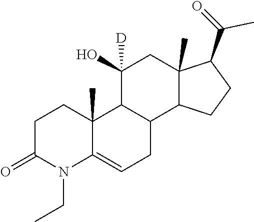 [SMILES: [2H][C@]1(O)C[C@@]2(C)C(CC[C@@H]2C(C)=O)C2CC=C3N(CC)C(=O)CC[C@]3(C)C21] | (4aR,5S,6aS,7S)-7-acetyl-1- ethyl-5-hydroxy-4a,6a-dimethyl- 1,3,4,4a,4b,5,6,6a,7,8,9,9a,9b,10- tetradecahydro-2H-indeno[5,4- f]quinolin-2-one-5-d |
| 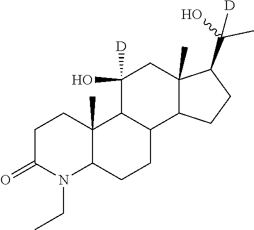 [SMILES: [2H]C(C)(O)[C@H]1CCC2C3CCC4N(CC)C(=O)CC[C@]4(C)C3[C@@]([2H])(O)C[C@@]21C] | (4aR,5S,6aS,7S)-1-ethyl-5- hydroxy-7-(1-hydroxyethyl- 1-d)-4a,6a-dimethylhexadeca- hydro-2H-indeno[5,4-f]quinolin- 2-one-5-d |
| 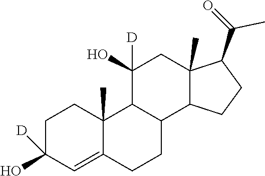 [SMILES: [2H][C@]1(O)C[C@@]2(C)C(CC[C@@H]2C(C)=O)C2CCC3=C[C@@]([2H])(O)CC[C@]3(C)C21] | 1-((3S,10R,11S,13S,17S)-3,11- dihydroxy-10,13-dimethyl- 2,3,6,7,8,9,10,11,12,13,14,15,16,17- tetradecahydro-1H-cyclopenta- [a]phenanthren-17-yl-3,11-d2)- ethan-1-one |
| 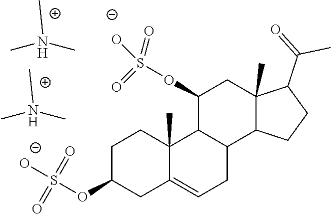 | (3S,10R,11S,13S)-17- acetyl-10,13-dimethyl- 2,3,4,7,8,9,10,11,12,13,14,15,16,17- tetradecahydro-1H-cyclopenta- [a]phenanthrene-3,11-diyl bis- (sulfate)trimethylammonium salt |
| 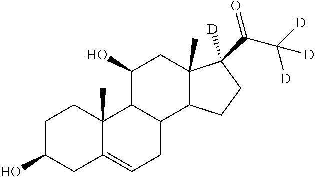 [SMILES: [2H]C([2H])([2H])C(=O)[C@@]1([2H])CCC2C3CC=C4C[C@@H](O)CC[C@]4(C)C3[C@@H](O)C[C@@]21C] | 1-((3S,10R,11S,13S,17S)-3,11- dihydroxy-10,13-dimethyl- 2,3,4,7,8,9,10,11,12,13,14,15,16,17- tetradecahydro-1H-cyclopenta- [a]phenanthren-17-yl-17-d)ethan- 1-one-2,2,2-d3 |
| 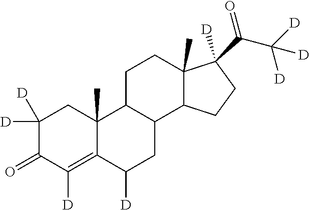 [SMILES: [2H]C1=C2C([2H])CC3C(CC[C@@]4(C)C3CC[C@]4([2H])C(=O)C([2H])([2H])[2H])[C@@]2(C)CC([2H])([2H])C1=O] | (10R,11S,13S,17S)-17- (acetyl-d3)-10,13-dimethyl- 1,2,6,7,8,9,10,11,12,13,14,15,16,17- tetradecahydro-3H-cyclopenta- [a]phenanthren-3-one-2,2,4,6,17-d5 |
| 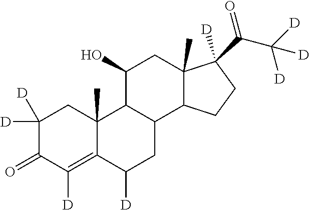 [SMILES: [2H]C1=C2C([2H])CC3C([C@@H](O)C[C@@]4(C)C3CC[C@]4([2H])C(=O)C([2H])([2H])[2H])[C@@]2(C)CC([2H])([2H])C1=O] | (10R,11S,13S,17S)-17-(acetyl- d3)-11-hydroxy-10,13-dimethyl- 1,2,6,7,8,9,10,11,12,13,14,15,16,17- tetradecahydro-3H-cyclopenta[a]- phenanthren-3-one-2,2,4,6,17-d5 |

or a pharmaceutically acceptable salt, stereoisomer or tautomer thereof.
11. A pharmaceutical composition comprising a compound of any one of embodiments 1-10, a pharmaceutically acceptable salt, stereoisomer or tautomer thereof, and a pharmaceutically acceptable carrier.
12. A compound, or a pharmaceutically acceptable salt, stereoisomer or tautomer thereof, of any one of embodiments 1-10 for use in AMPK activation.

Certain specific aspects and embodiments will be explained in more detail with reference to the following examples, which are provided only for purposes of illustration and should not be construed as limiting the scope in any manner.

### EXAMPLES

### Example 1: Preparation of Compounds of Formula (II)

Compounds described herein may be synthesized according to the methods used in Scheme 3.

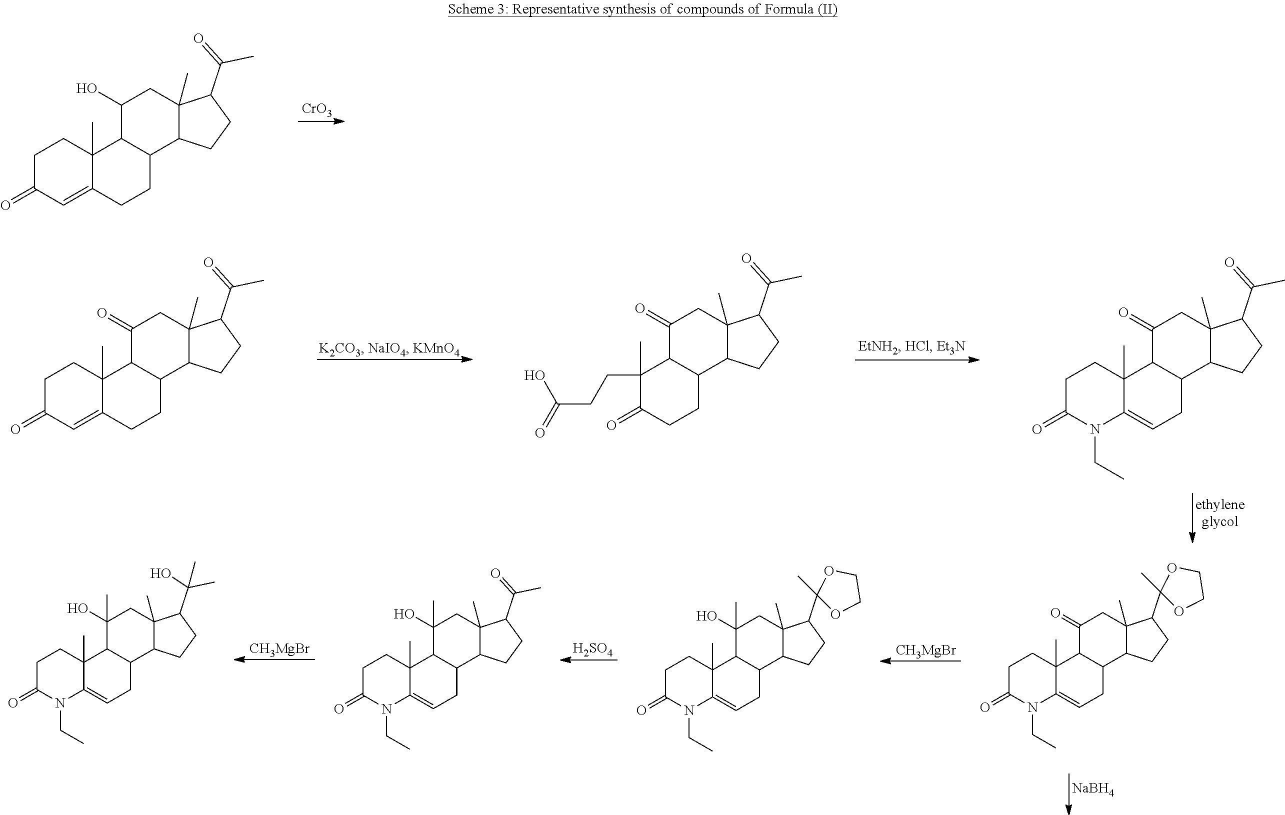

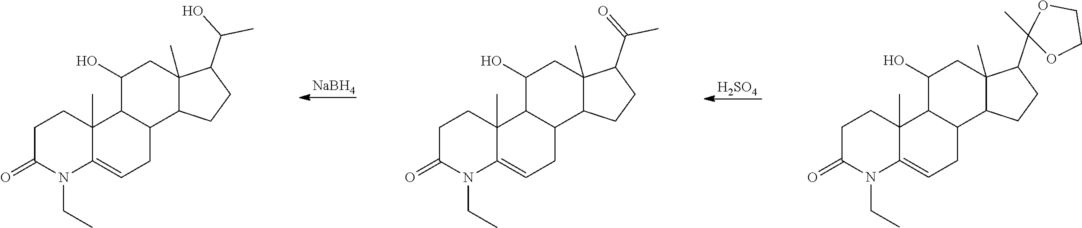

### Example 2: PGC-1α Assay

The compounds of Table 2 were synthesized and tested in a PGC-1α assay.

Culture conditions: DMEM containing 25 mM glucose with 10% FBS supplementation; grown at 37° C. and 5% CO₂.

Assay Conditions: C2C12 cells were cultured in DMEM supplemented in 10% FBS up to 80% confluence. The cells were further trypsinized and seeded into a 96 well plate at a density of 5000 cells/well (well volume—100 μL) (Day 0). Following attachment, the cells were allowed to reach 80-90% confluence in the plate (typically 48 hours post seeding) and further differentiated using DMEM+2% Horse Serum (200 μL/well). The media was changed every day or at least every alternate day for 5 days to allow myoblasts to differentiate into myotubes. On day 7 (post seeding), the cells were treated with standards and test compounds for PGC-1α assay.

PGC-1α assay: For the detection of PGC-1α, the cells were incubated with 0.5 g/ml primary antibody (Merck, WH10010891M3) in PBS-T containing 5% BSA at 4° C. overnight. The cells were then washed three times with PBS-T for 5 minutes and incubated with 1:1000 dilution secondary antibody (Anti-rabbit IgG, HRP-linked Antibody, Cell Signaling) in PBS-T with 100% BSA for 1 hour at RT. Cells were washed three times with PBS-T for 5 minutes The cells were incubated with 100 μl TMB substrate solution for 30 minutes and the reaction was stopped with 100 μl of 2N H₂SO₄. Then the plate was read at 450 nM using ELISA plate reader and absorbance recorded. 0 activity was calculated using DMSO control as 100%. (For plate optical density calculations, a background correction was taken at 540 nm). Each compound was tested at a concentration of 0.01 nM. The results of the assay are presented in Table 2.

TABLE 2
PGC1-Alpha Assay

| | | | Single point |
|---|---|---|---|
| | | Single point | PGC1-Alpha |
| | Single point | PGC1-Alpha | Assay: |
| | PGC1-Alpha | Assay: | Average |
| | Assay: | Average | Activity (%) |
| Compound | Activity (%) | Activity (%) | Count |
| 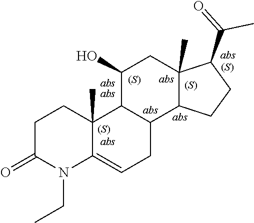 | 116 | 116 | 1 |
| 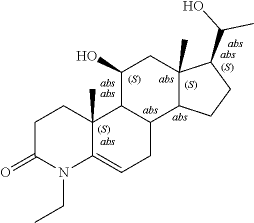 | 94 | 94 | 1 |
| 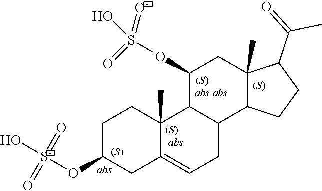 | 101 | 101 | 1 |
| 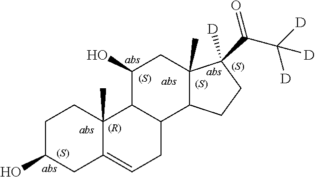 | Exp. 1: 141 Exp. 2: 106 Exp. 3: 99 | 115 | 3 |
| 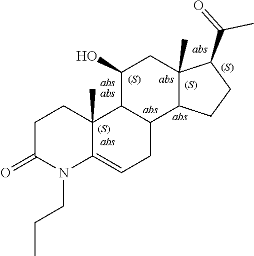 | 109 | 109 | 1 |
| 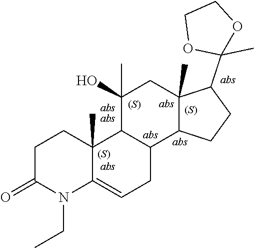 | 119 | 119 | 1 |
| 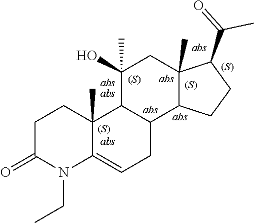 | 118 | 118 | 1 |
| 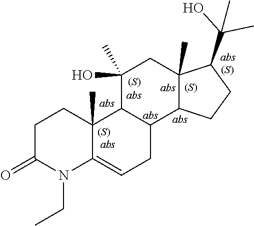 | 126 | 126 | 1 |
| 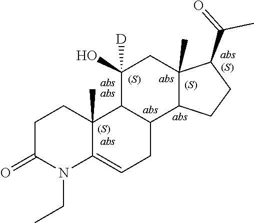 | Exp. 1: 137 Exp. 2: 112 Exp. 3: 131 | 127 | 3 |

1. A compound of Formula (II), 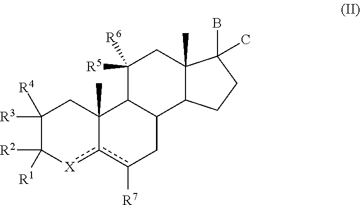
or a pharmaceutically acceptable salt, stereoisomer or tautomer thereof, wherein: [symbol]
is independently either a single bond or a double bond, provided that adjacent double bonds are not allowed;
R¹ is ═O, OH or OSO₃H
R² is absent, H or deuterium;
R³ and R⁴ are independently H or deuterium;
R⁵ is OH, H or OSO₃H
R⁶ is H, deuterium or CH₃;
R⁷ is H or deuterium;
X is CH, CD, CH₂ or NR⁸, wherein R⁸ is C₁-C₆ alkyl;
B is selected from the group consisting of —COR⁹, C₁-C₆ alkyl, and 5-6 membered heterocycloalkyl, wherein R⁹ is CH₃ or CD₃, the C₁-C₆ alkyl is further substituted with OH, D or both, and the 5-6 membered heterocycloalkyl is optionally substituted with CH₃; and
C is H or deuterium;
provided that when X is CH, CD, or CH₂, then B is —COR⁹, wherein R⁹ is CH₃ or CD₃, and the compound is not selected from the group consisting of:
1-((3S,8S,9S,10R,11S,13S,14S,17S)-3,11-dihydroxy-10,13-dimethyl-2,3,4,7,8,9,10,11,12,13,14,15,16,17-tetradecahydro-1H-cyclopenta[a]phenanthren-17-yl)ethanone,
1-((3S,8S,9S,10R,11S,13S,14S,17S)-3,11-dihydroxy-10,13-dimethyl-2,3,6,7,8,9,10,11,12,13,14,15,16,17-tetradecahydro-1H-cyclopenta[a]phenanthren-17-yl)ethanone,
(8S,9S,10R,11S,13S,14S,17S)-17-acetyl-11-hydroxy-10,13-dimethyl-6,7,8,9,10,11,12,13,14,15,16,17-dodecahydro-1H-cyclopenta[a]phenanthren-3(2H)-one,
1-((3S,8S,9S,10S,11S,13S,14S,17S)-3,11-dihydroxy-10,13-dimethylhexadecahydro-1H-cyclopenta[a]phenanthren-17-yl)ethanone,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl)ethan-1-one,
(10R,11S,13S,17S)-17-acetyl-11-hydroxy-10,13-dimethyl-1,2,6,7,8,9,10,11,12,13,14,15,16,17-tetradecahydro-3H-cyclopenta[a]phenanthren-3-one,
(10S,11S,13S,17S)-17-acetyl-11-hydroxy-10,13-dimethylhexadecahydro-3H-cyclopenta[a]phenanthren-3-one,
(10R,11S,13S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10S,11S,13S,17S)-3,11-dihydroxy-10,13-dimethylhexadecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3-d)ethan-1-one,
1-((3S,10R,11S,13S,17S)-3,11-dihydroxy-10,11,13-trimethyl-2,3,4,7,8,9,10,11,12,13,14,15,16,17-tetradecahydro-1H-cyclopenta[a]phenanthren-17-yl)ethan-1-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-11-d)ethan-1-one,
(8S,9S,10R,11S,13S,14S,17S)-17-acetyl-11-hydroxy-10,11,13-trimethyl-1,2,6,7,8,9,10,11,12,13,14,15,16,17-tetradecahydro-3H-cyclopenta[a]phenanthren-3-one,
1-((3S,10R,11S,13S,17S)-3,11-dihydroxy-10,13-dimethyl-2,3,4,7,8,9,10,11,12,13,14,15,16,17-tetradecahydro-1H-cyclopenta[a]phenanthren-17-yl-3,11-d₂)ethan-1-one,
1-((8S,9S,10R,11S,13 S,14S)-3,11-dihydroxy-10,13-dimethyl-2,3,4,7,8,9,10,11,12,13,14,15,16,17-tetradecahydro-1H-cyclopenta[a]phenanthren-17-yl)ethanone,
(8S,9S,10R,11S,13S,14S)-17-acetyl-11-hydroxy-10,13-dimethyl-6,7,8,9,10,11,12,13,14,15,16,17-dodecahydro-1H-cyclopenta[a]phenanthren-3(2H)-one,
(3S,10S,11S,13S,17S)-17-((R)-1-hydroxyethyl)-10,13-dimethylhexadecahydro-1H-cyclopenta[a]phenanthrene-3,11-diol,
(3S,10R,11S,13S,17S)-17-((R)-1-hydroxyethyl)-10,13-dimethyl-2,3,6,7,8,9,10,11,12,13,14,15,16,17-tetradecahydro-1H-cyclopenta[a]phenanthrene-3,11-diol,
(10R,11S,13S,17S)-11-hydroxy-17-((R)-1-hydroxyethyl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxypropan-2-yl)-10,13-dimethyl-1,2,6,7,8,9,10,11,12,13,14,15,16,17-tetradecahydro-3H-cyclopenta[a]phenanthren-3-one,
(10R,11S,13S,17S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one,
(3S,10R,11S,13S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthrene-3,11-diol,
(3S,10R,11S,13S,17S)-17-((R)-1-hydroxyethyl)-10,13-dimethyl-2,3,4,7,8,9,10,11,12,13,14,15,16,17-tetradecahydro-1H-cyclopenta[a]phenanthrene-3,11-diol,
(3S,10R,11S,13R,17S)-17-ethyl-10,13-dimethyl-2,3,4,7,8,9,10,11,12,13,14,15,16,17-tetradecahydro-1H-cyclopenta[a]phenanthrene-3,11-diol, and
Trimethylammonium (3S, 8S, 9S, 10R,11S,13S, 17S)-17-acetyl-11-hydroxy-10,13-dimethyl-2,3,4,7,8,9,10,11,12,13,14,15,16,17-tetradecahydro-1H-cyclopenta[a]phenanthren-3-yl sulfate,
or a pharmaceutically acceptable salt, stereoisomer or tautomer thereof.

2. The compound of claim 1, or a pharmaceutically acceptable salt, stereoisomer or tautomer thereof, wherein X is NR⁸, wherein R⁸ or C₁-C₆ alkyl.

3. The compound of claim 1, or a pharmaceutically acceptable salt, stereoisomer or tautomer thereof, wherein X is CH, CD, or CH₂.

4. The compound of claim 1, or a pharmaceutically acceptable salt, stereoisomer or tautomer thereof, wherein R¹ is ═O and R² is absent.

5. The compound of claim 1, or a pharmaceutically acceptable salt, stereoisomer or tautomer thereof, wherein R³ and R⁴ are H.

6. The compound of claim 1, or a pharmaceutically acceptable salt, stereoisomer or tautomer thereof, wherein R⁵ is OH.

7. The compound of claim 1, or a pharmaceutically acceptable salt, stereoisomer or tautomer thereof, wherein R⁶ is H.

8. The compound of claim 1, or a pharmaceutically acceptable salt, stereoisomer or tautomer thereof, wherein B is —COR⁹, wherein R⁹ is CH₃ or CD₃.

9. The compound of claim 1, or a pharmaceutically acceptable salt, stereoisomer or tautomer thereof, wherein the compound is of formula (II-a):

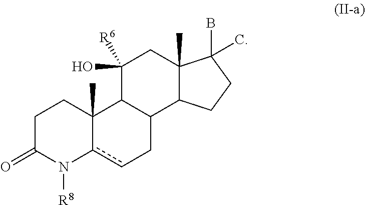

10. A compound, or a pharmaceutically acceptable salt, stereoisomer or tautomer thereof, wherein the compound is selected from the group consisting of:

| Compound | Name |
|---|---|
| 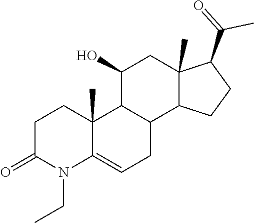 [SMILES: CCN1C(=O)CC[C@@]2(C)C1=CCC1C2[C@@H](O)C[C@@]2(C)C1CC[C@@H]2C(C)=O] | (4aR,5S,6aS,7S)-7-acetyl-1-ethyl- 5-hydroxy-4a,6a-dimethyl- 1,3,4,4a,4b,5,6,6a,7,8,9,9a,9b,10- tetradecahydro-2H-indeno[5,4-f]- quinolin-2-one |
| 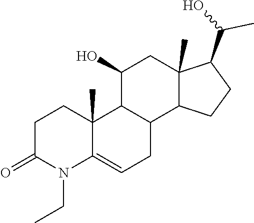 [SMILES: CCN1C(=O)CC[C@@]2(C)C1=CCC1C2[C@@H](O)C[C@@]2(C)C1CC[C@@H]2C(C)O] | (4aR,5S,6aS,7S)-1-ethyl-5- hydroxy-7-((R)-1-hydroxy- ethyl)-4a,6a-dimethyl- 1,3,4,4a,4b,5,6,6a,7,8,9,9a,9b,10- tetradecahydro-2H-indeno[5,4-f]- quinolin-2-one |
| 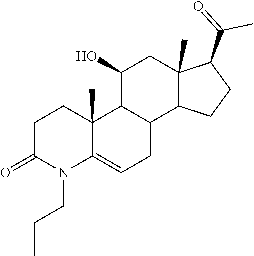 [SMILES: CCCN1C(=O)CC[C@@]2(C)C1=CCC1C2[C@@H](O)C[C@@]2(C)C1CC[C@@H]2C(C)=O] | (4aR,5S,6aS,7S)-7-acetyl-5- hydroxy-4a,6a-dimethyl-1-propyl- 1,3,4,4a,4b,5,6,6a,7,8,9,9a,9b,10- tetradecahydro-2H-indeno[5,4-f]- quinolin-2-one |
| 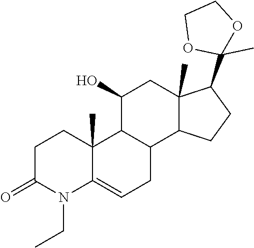 [SMILES: CCN1C(=O)CC[C@@]2(C)C1=CCC1C2[C@@H](O)C[C@@]2(C)C1CC[C@@H]2C1(C)OCCO1] | (4aR,5S,6aS,7S)-1-ethyl-5- hydroxy-4a,6a-dimethyl-7-(2- methyl-1,3-dioxolan-2-yl)- 1,3,4,4a,4b,5,6,6a,7,8,9,9a,9b,10- tetradecahydro-2H-indeno[5,4-f]- quinolin-2-one |
| 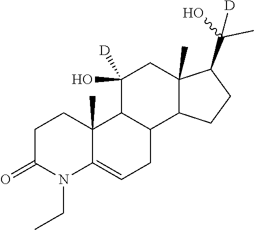 [SMILES: [2H]C(C)(O)[C@H]1CCC2C3CC=C4N(CC)C(=O)CC[C@]4(C)C3[C@@]([2H])(O)C[C@@]21C] | (4aR,5S,6aS,7S)-1-ethyl-5- hydroxy-7-(1-hydroxyethyl- 1-d)-4a,6a-dimethyl- 1,3,4,4a,4b,5,6,6a,7,8,9,9a,9b,10- tetradecahydro-2H-indeno- [5,4-f]quinolin-2-one-5-d |
| 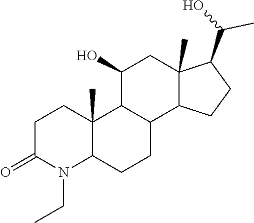 [SMILES: CCN1C(=O)CC[C@]2(C)C3C(CCC12)C1CC[C@H](C(C)O)[C@@]1(C)C[C@@H]3O] | (4aR,5S,6aS,7S)-1-ethyl-5- hydroxy-7-((R)-1-hydroxyethyl)- 4a,6a-dimethylhexadecahydro- 2H-indeno[5,4-f]quinolin-2-one |
| 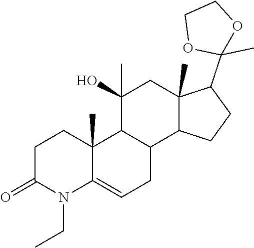 [SMILES: CCN1C(=O)CC[C@@]2(C)C1=CCC1C3CCC(C4(C)OCCO4)[C@@]3(C)C[C@](C)(O)C12] | (4aR,5S,6aS)-1-ethyl-5- hydroxy-4a,5,6a-trimethyl-7- (2-methyl-1,3-dioxolan-2-yl)- 1,3,4,4a,4b,5,6,6a,7,8,9,9a,9b,10- tetradecahydro-2H-indeno[5,4-f]- quinolin-2-one |
| 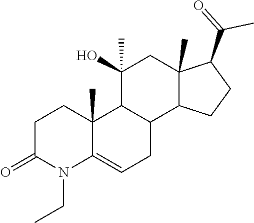 [SMILES: CCN1C(=O)CC[C@@]2(C)C1=CCC1C2[C@@](C)(O)C[C@@]2(C)C1CC[C@@H]2C(C)=O] | (4aR,5S,6aS,7S)-7-acetyl-1-ethyl- 5-hydroxy-4a,5,6a-trimethyl- 1,3,4,4a,4b,5,6,6a,7,8,9,9a,9b,10- tetradecahydro-2H-indeno[5,4-f]- quinolin-2-one |
| 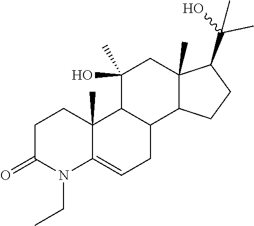 [SMILES: CCN1C(=O)CC[C@@]2(C)C1=CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan- 2-yl)-4a,5,6a-trimethyl- 1,3,4,4a,4b,5,6,6a,7,8,9,9a,9b,10- tetradecahydro-2H-indeno- [5,4-f]quinolin-2-one |
| 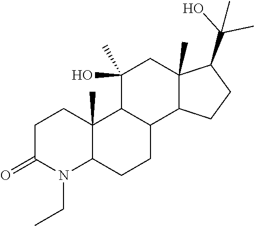 [SMILES: CCN1C(=O)CC[C@@]2(C)C1CCC1C2[C@@](C)(O)C[C@@]2(C)C1CC[C@@H]2C(C)(C)O] | (4aR,5S,6aS,7S)-1-ethyl-5- hydroxy-7-(2-hydroxypropan-2- yl)-4a,5,6a-trimethylhexadeca- hydro-2H-indeno[5,4-f]quinolin- 2-one |
| 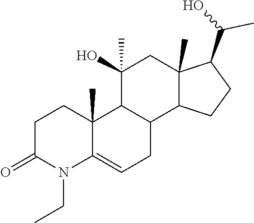 [SMILES: CCN1C(=O)CC[C@@]2(C)C1=CCC1C2[C@@](C)(O)C[C@@]2(C)C1CC[C@@H]2C(C)O] | (4aR,5S,6aS,7S)-1-ethyl-5- hydroxy-7-((R)-1-hydroxy- ethyl)-4a,5,6a-trimethyl- 1,3,4,4a,4b,5,6,6a,7,8,9,9a,9b,10- tetradecahydro-2H-indeno- [5,4-f]quinolin-2-one |
| 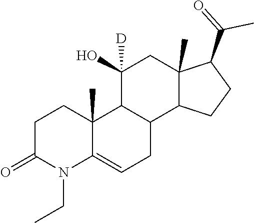 [SMILES: [2H][C@]1(O)C[C@@]2(C)C(CC[C@@H]2C(C)=O)C2CC=C3N(CC)C(=O)CC[C@]3(C)C21] | (4aR,5S,6aS,7S)-7-acetyl-1-ethyl- 5-hydroxy-4a,6a-dimethyl- 1,3,4,4a,4b,5,6,6a,7,8,9,9a,9b,10- tetradecahydro-2H-indeno[5,4-f]- quinolin-2-one-5-d |
| 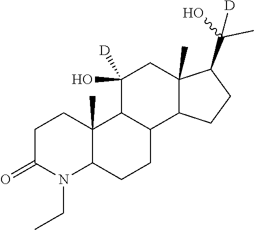 [SMILES: [2H]C(C)(O)[C@H]1CCC2C3CCC4N(CC)C(=O)CC[C@]4(C)C3[C@@]([2H])(O)C[C@@]21C] | (4aR,5S,6aS,7S)-1-ethyl-5- hydroxy-7-(1-hydroxyethyl-1-d)- 4a,6a-dimethylhexadecahydro- 2H-indeno[5,4-f]quinolin-2-one- 5-d |
| 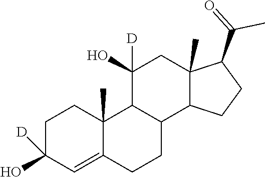 [SMILES: [2H][C@]1(O)C[C@@]2(C)C(CC[C@@H]2C(C)=O)C2CCC3=C[C@@]([2H])(O)CC[C@]3(C)C21] | 2,3,6,7,8,9,10,11,12,13,14,15,16,17- tetradecahydro-1H-cyclopenta- [a]phenanthren-17-yl-3,11-d2)- ethan-1-one |
| 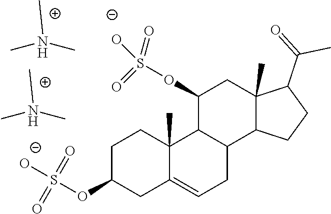 | (3S,10R,11S,13S)-17- acetyl-10,13-dimethyl- 2,3,4,7,8,9,10,11,12,13,14,15,16,17- tetradecahydro-1H-cyclopenta- [a]phenanthrene-3,11-diyl bis- (sulfate)trimethylammonium salt |
| 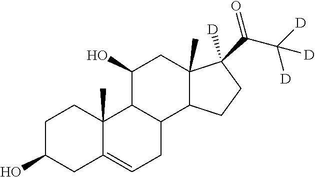 [SMILES: [2H]C([2H])([2H])C(=O)[C@@]1([2H])CCC2C3CC=C4C[C@@H](O)CC[C@]4(C)C3[C@@H](O)C[C@@]21C] | 1-((3S,10R,11S,13S,17S)-3,11- dihydroxy-10,13-dimethyl- 2,3,4,7,8,9,10,11,12,13,14,15,16,17- tetradecahydro-1H-cyclopenta[a]- phenanthren-17-yl-17-d)ethan-1- one-2,2,2-d3 |
| 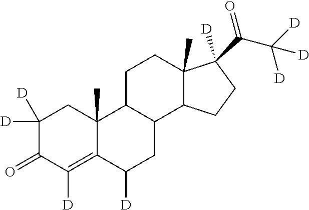 [SMILES: [2H]C1=C2C([2H])CC3C(CC[C@@]4(C)C3CC[C@]4([2H])C(=O)C([2H])([2H])[2H])[C@@]2(C)CC([2H])([2H])C1=O] | (10R,13S,17S)-17-(acetyl- d3)-10,13-dimethyl- 1,2,6,7,8,9,10,11,12,13,14,15,16,17- tetradecahydro-3H-cyclopenta[a]- phenanthren-3-one-2,2,4,6,17-d5 |
| 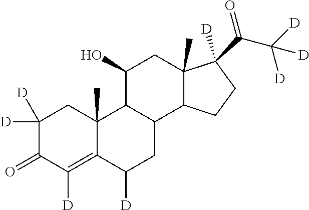 [SMILES: [2H]C1=C2C([2H])CC3C([C@@H](O)C[C@@]4(C)C3CC[C@]4([2H])C(=O)C([2H])([2H])[2H])[C@@]2(C)CC([2H])([2H])C1=O] | (10R,11S,13S,17S)-17-(acetyl- d3)-11-hydroxy-10,13-dimethyl- 1,2,6,7,8,9,10,11,12,13,14,15,16,17- tetradecahydro-3H-cyclopenta[a]- phenanthren-3-one-2,2,4,6,17-d5 |

or a pharmaceutically acceptable salt, stereoisomer or tautomer thereof.

11. A pharmaceutical composition comprising a compound of claim 1, a pharmaceutically acceptable salt, stereoisomer or tautomer thereof, and a pharmaceutically acceptable carrier.

12. A compound, or a pharmaceutically acceptable salt, stereoisomer or tautomer thereof, of claim 1 for use in AMPK activation.
